(19) United States
(12) Patent Application Publication
(10) Pub. No.: US 2024/0123239 A1
(43) Pub. Date: Apr. 18, 2024

(54) HEAT REDUCTION ASSOCIATED WITH PROSTHESES

(71) Applicant: COCHLEAR LIMITED, Macquarie University, NSW (AU)

(72) Inventors: Helmut Christian EDER, Macquarie University (AU); Dirk Arno FIEDLER, Macquarie University (AU); Guilhem Yvan VAVELIN, Macquarie University (AU); Werner MESKENS, Macquarie University (AU)

(21) Appl. No.: 18/274,519

(22) PCT Filed: Jan. 27, 2022
(86) PCT No.: PCT/IB2022/050726

§ 371 Date: Jul. 27, 2023

Related U.S. Application Data
(60) Provisional application No. 63/142,256, filed on Jan. 27, 2021.

(51) Int. Cl. A61N 1/375; A61N 1/36; A61N 1/378

(52) U.S. Cl. CPC A61N 1/375; A61N 1/36038; A61N 1/36046; A61N 1/3787

(57) ABSTRACT

A method, including placing a transcutaneous power transfer apparatus at a location on a surface of the skin proximate an implanted medical device, transferring power from the apparatus to the implanted medical device, and actively cooling the transcutaneous power transfer apparatus below the ambient temperature prior to and/or after commencing transfer of power from the apparatus to the implanted medical device.

### CROSS-REFERENCE TO RELATED APPLICATIONS

This application claims priority to U.S. Provisional Application No. 63/142,256, entitled HEAT REDUCTION ASSOCIATED WITH PROSTHESES, filed on Jan. 27, 2021, naming Helmut Christian EDER of Macquarie University, Australia as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In accordance with an exemplary embodiment, there is a method, comprising: placing a transcutaneous power transfer apparatus at a location on a surface of the skin proximate an implanted medical device; transferring power from the apparatus to the implanted medical device; and actively cooling the transcutaneous power transfer apparatus below the ambient temperature prior to and/or after commencing transfer of power from the apparatus to the implanted medical device.

In accordance with another exemplary embodiment, there is a method, comprising: obtaining a device configured to transcutaneously charge and/or power an implanted prosthesis implanted in a recipient, which device has a rechargeable power storage component from which power is extracted to charge and/or power the implanted prosthesis, the power storage device having a state of charge less than fully charged; recharging the power storage component to elevate the state of charge of the power storage component; and at least one of before or after the action of recharging, chilling the device so that an outer surface of the device that interfaces with skin of a person during charging and/or powering of the implanted prosthesis has a temperature that is lower than that which would otherwise be the case in the absence of the chilling.

In accordance with another exemplary embodiment, there is a device, comprising: an inductive power transmission system configured to transfer power to an implanted medical device; a skin interface surface; and a dedicated passive conduction thermal transfer apparatus configured for temperature management of the device during the transfer of power.

In accordance with another exemplary embodiment, there is a method, comprising: placing a transcutaneous power transfer apparatus at a location on a surface of skin of a life human proximate an implanted medical device; transcutaneous transferring power from the apparatus to the implanted medical device; and at least partially recharging an implanted battery of the implanted medical device by increasing a charge of the battery by at least 10 mAh within 10 minutes using the transferred power.

In accordance with another exemplary embodiment, there is a device, comprising: a battery charging apparatus; and a cooling device, wherein the device is a dedicated prosthesis component charging device configured to recharge a power storage portion of the prosthesis component before and/or after cooling an assembly of which the power storage portion is apart using the cooling device.

In accordance with another embodiment, there is a headpiece of a hearing prosthesis, comprising: a DC battery; an inductive power driver including transistors, the inductive power driver being configured to use the transistors to convert the direct current of the battery to alternating current; a magnet; an inductive coil extending about the magnet, wherein the inductive coil is in electrical communication with the inductive power driver so that the inductive coil receives the alternating current and generates an inductance field to power an implantable hearing prosthesis; and a dedicated passive conduction thermal transfer apparatus configured for temperature management of the headpiece during the generation of the inductance field to power the implantable hearing prosthesis, wherein the dedicated passive conduction thermal transfer apparatus is a dedicated thermal mass made of metal configured for thermal mass cooling of the headpiece.

### BRIEF DESCRIPTION OF THE DRAWINGS

Embodiments of the present invention are described below with reference to the attached drawings, in which:

FIG. 1 is a perspective view of an exemplary hearing prosthesis in which at least some of the teachings detailed herein are applicable;

FIG. 1A is a view of an exemplary sight prosthesis in which at least some of the teachings herein are applicable;

FIG. 2 schematically illustrates another exemplary hearing prosthesis in which at least some of the teachings detailed herein are applicable;

FIG. 3 schematically illustrates another exemplary hearing prosthesis in which at least some of the teachings detailed herein are applicable;

FIG. 4 schematically illustrates another exemplary hearing prosthesis in which at least some of the teachings detailed herein are applicable;

FIG. 5 schematically illustrates another exemplary hearing prosthesis in which at least some of the teachings detailed herein are applicable;

FIG. 6 schematically illustrates another exemplary hearing prosthesis in which at least some of the teachings detailed herein are applicable;

FIGS. 7-9 depict functional diagrams associated with some embodiments;

FIG. 10 is a cross-section of an apparatus used in some embodiments;

FIG. 11 schematically illustrates additional exemplary hearing prostheses in which at least some of the teachings detailed herein are applicable;

FIG. 12 presents an exemplary flowchart for an exemplary method;

FIG. 13 presents a diagram of a head of a human;

FIGS. 14 and 25 depict exemplary charging devices for prosthesis devices.

FIGS. 15 and 24 present respective exemplary flowcharts for exemplary methods; and

FIGS. 16-23 presents additional exemplary devices according to exemplary embodiments.

### DETAILED DESCRIPTION

Merely for ease of description, the techniques presented herein are primarily described herein with reference to an illustrative medical device, namely a cochlear implant. However, it is to be appreciated that the techniques presented herein may also be used with a variety of other medical devices that, while providing a wide range of therapeutic benefits to recipients, patients, or other users, may benefit from the teachings herein used in other medical devices. For example, any techniques presented herein described for one type of hearing prosthesis, such as a cochlear implant, corresponds to a disclosure of another embodiment of using such teaching with another hearing prostheses, including bone conduction devices (percutaneous, active transcutaneous and/or passive transcutaneous), middle ear auditory prostheses, direct acoustic stimulators, and also utilizing such with other electrically simulating auditory prostheses (e.g., auditory brain stimulators), etc. The techniques presented herein can be used with implantable/implanted microphones, whether or not used as part of a hearing prosthesis (e.g., a body noise or other monitor, whether or not it is part of a hearing prosthesis). The techniques presented herein can also be used with vestibular devices (e.g., vestibular implants), sensors, seizure devices (e.g., devices for monitoring and/or treating epileptic events, where applicable), sleep apnea devices, electroporation, etc., and thus any disclosure herein is a disclosure of utilizing such devices with the teachings herein, providing that the art enables such. It is also noted that in an exemplary embodiment, the teachings herein can be used with a retinal implant device. Thus, any disclosure herein corresponds to a disclosure of expanding functionality to include the functionality of a retinal implant, and, for example, any disclosure of a cochlear implant processor corresponds to a light processor. In further embodiments, the techniques presented herein may be used with air purifiers or air sensors (e.g., automatically adjust depending on environment), hospital beds, identification (ID) badges/bands, or other hospital equipment or instruments, where such rely upon behind the ear devices.

By way of example, any of the technologies detailed herein which are associated with components that are implanted in a recipient can be combined with information delivery technologies disclosed herein, such as for example, devices that evoke a hearing percept and/or devices that evoke a vision percept, to convey information to the recipient. By way of example only and not by way of limitation, a sleep apnea implanted device can be combined with a device that can evoke a hearing percept so as to provide information to a recipient, such as status information, etc. In this regard, the various sensors detailed herein and the various output devices detailed herein can be combined with such a non-sensory prosthesis or any other nonsensory prosthesis that includes implantable components so as to enable a user interface as will be described herein that enables information to be conveyed to the recipient, which information is associated with the implant.

Moreover, embodiments need not necessarily provide input or status information to the recipient. Instead, the various sensors detailed herein can be utilized in combination with the nonsensory implants so as to enable control or performance adjustments of the implanted component. For example, the embodiments that utilize sensors and the associated logic circuitry that would be combined with a sleep apnea device, for example, can be utilized to enable the recipient to input commands to control the implant. Such can potentially also be done with respect to a bionic arm or bionic leg, etc. In this regard, embodiments can enable a user interface that can enable a recipient to provide input into the prosthesis to control the prosthesis without utilizing any artificial external component. For example, embodiments can enable the input utilizing only the recipient's voice and/or only the recipient's hand/fingers. Thus, embodiments can enable control of such prostheses utilizing only a recipient's hand and/or only a recipient's voice. Accordingly, at least some exemplary embodiments can combine hearing prosthesis technology with the innovations detailed herein with other implant technologies to enable control without the need of other artificial devices.

Thus, the teachings detailed herein are implemented in sensory prostheses, such as hearing devices, including hearing implants specifically, and neural stimulation devices in general. Other types of sensory prostheses can include retinal implants. Accordingly, any teaching herein with respect to a sensory prosthesis corresponds to a disclosure of utilizing those teachings in/with a hearing implant and in/with a retinal implant, unless otherwise specified, providing the art enables such. To be clear, any teaching herein with respect to a specific sensory prosthesis corresponds to a disclosure of utilizing those teachings in/with any of the aforementioned hearing prostheses, and vice versa. Corollary to this is at least some teachings detailed herein can be implemented in somatosensory implants and/or chemosensory implants. Accordingly, any teaching herein with respect to a sensory prosthesis corresponds to a disclosure of utilizing those teachings with/in a somatosensory implant and/or a chemosensory implant.

While the teachings detailed herein will be described for the most part with respect to hearing prostheses, in keeping with the above, it is noted that any disclosure herein with respect to a hearing prosthesis corresponds to a disclosure of another embodiment of utilizing the associated teachings with respect to any of the other prostheses noted herein, whether a species of a hearing prosthesis, or a species of a sensory prosthesis, such as a retinal prosthesis. In this regard, any disclosure herein with respect to evoking a hearing percept corresponds to a disclosure of evoking other types of neural percepts in other embodiments, such as a visual/sight percept, a tactile percept, a smell precept or a taste percept, unless otherwise indicated and/or unless the art does not enable such. Any disclosure herein of a device, system, and/or method that is used to or results in ultimate stimulation of the auditory nerve corresponds to a disclosure of an analogous stimulation of the optic nerve utilizing analogous components/methods/systems. All of this can be separately or in combination.

Embodiments detailed herein focus on the utilization of a hearing prosthesis to provide status and information a recipient. It is to be understood that in some embodiments, a retinal prosthesis can be utilized to provide visual input to the recipient. By way of example only and not by way of limitation, in an exemplary embodiment, the retinal prosthesis can be configured to results in a vision of an artificial image, which can correspond to words or the like, which can correspond to a status of the prostheses. Accordingly, any disclosure herein associated with providing sound-based or hearing percept base information the recipient also corresponds to a disclosure of providing vision based information to the recipient and vice versa.

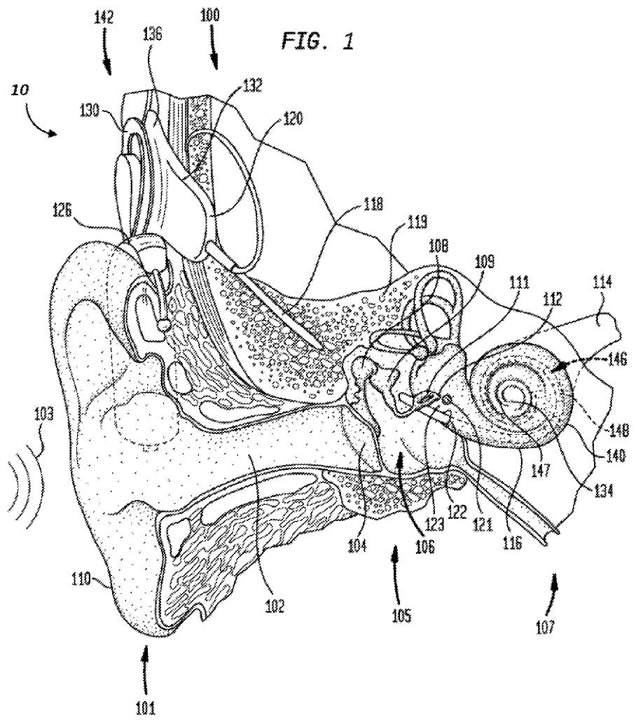

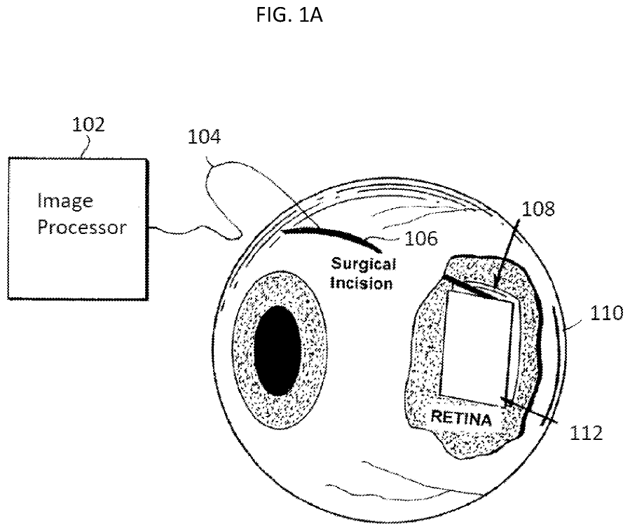

FIG. 1 is a perspective view of a totally implantable cochlear implant, referred to as cochlear implant 100, implanted in a recipient, to which some embodiments detailed herein and/or variations thereof are applicable. The totally implantable cochlear implant 100 is part of a system 10 that can include external components, in some embodiments, as will be detailed below. It is noted that the teachings detailed herein are applicable, in at least some embodiments, to any type of hearing prosthesis having an implantable microphone. The teachings detailed herein are also applicable, in at least some embodiments, to any type of hearing prosthesis not having an implantable microphone, and thus are applicable to non-totally implantable hearing prostheses.

It is noted that in alternate embodiments, the teachings detailed herein and/or variations thereof can be applicable to other types of hearing prostheses, such as, for example, bone conduction devices (e.g., active transcutaneous bone conduction devices), Direct Acoustic Cochlear Implant (DACI), etc. Embodiments can include any type of hearing prosthesis that can utilize the teachings detailed herein and/or variations thereof. It is further noted that in some embodiments, the teachings detailed herein and/or variations thereof can be utilized by other types of prostheses beyond hearing prostheses.

The recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear channel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (not shown) where they are perceived as sound.

As shown, cochlear implant 100 comprises one or more components which are temporarily or permanently implanted in the recipient. Cochlear implant 100 is shown in FIG. 1 with an external device 142, that is part of system 10 (along with cochlear implant 100), which, as described below, is configured to provide power to the cochlear implant, where the implanted cochlear implant includes a battery that is recharged by the power provided from the external device 142. In the illustrative arrangement of FIG. 1, external device 142 can comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1 is merely illustrative, and other external devices may be used with embodiments of the present invention.

Cochlear implant 100 comprises an internal energy transfer assembly 132 which can be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Cochlear implant 100 further comprises a main implantable component 120 and an elongate electrode assembly 118. In some embodiments, internal energy transfer assembly 132 and main implantable component 120 are hermetically sealed within a biocompatible housing. In some embodiments, main implantable component 120 includes an implantable microphone assembly (not shown) and a sound processing unit (not shown) to convert the sound signals received by the implantable microphone in internal energy transfer assembly 132 to data signals. That said, in some alternative embodiments, the implantable microphone assembly can be located in a separate implantable component (e.g., that has its own housing assembly, etc.) that is in signal communication with the main implantable component 120 (e.g., via leads or the like between the separate implantable component and the main implantable component 120). In at least some embodiments, the teachings detailed herein and/or variations thereof can be utilized with any type of implantable microphone arrangement. Some additional details associated with the implantable microphone assembly 137 will be detailed below.

Main implantable component 120 further includes a stimulator unit (not shown) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments, electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, disposed along a length thereof. As noted, a stimulator unit generates stimulation signals which are applied by electrodes 148 to cochlea 140, thereby stimulating auditory nerve 114.

As noted above, cochlear implant 100 comprises a totally implantable prosthesis that is capable of operating, at least for a period of time, without the need for external device 142. Therefore, cochlear implant 100 can further comprise a rechargeable power source (not shown) that stores power received from external device 142. The power source can comprise, for example, a rechargeable battery. During operation of cochlear implant 100, the power stored by the power source is distributed to the various other implanted components as needed. The power source may be located in main implantable component 120, or disposed in a separate implanted location.

It is noted that the teachings detailed herein and/or variations thereof can be utilized with a non-totally implantable prosthesis. That is, in an alternate embodiment of the cochlear implant 100, the cochlear implant 100 is a traditional hearing prosthesis.

In some exemplary embodiments, a signal sent to the stimulator of the cochlear implant can be derived from an external microphone, in which case the system is called a semi-implantable device, or from an implanted microphone, which then refers to a fully implantable device. DACIs and other types of implants can also use an implanted microphone, and thus are also fully implantable devices. Fully implantable devices can have utility by presenting improved cosmesis, can have an improved immunity to certain noises (e.g., wind noise), can present few opportunities for loss or damage, and can at least sometimes be more resistant to clogging by debris or water, etc. DACIs can have utilitarian value by keeping the ear canal open, which can reduce the possibility of infection of the ear canal, which otherwise is humid, often impacted with cerumen (earwax), and irritated by the required tight fit of a non-implanted hearing aid.

FIG. 1A presents an exemplary embodiment of a neural prosthesis in general, and a retinal prosthesis and an environment of use thereof, in particular. In some embodiments of a retinal prosthesis, a retinal prosthesis sensor-stimulator 108 is positioned proximate the retina 110. In an exemplary embodiment, photons entering the eye are absorbed by a microelectronic array of the sensor-stimulator 108 that is hybridized to a glass piece 112 containing, for example, an embedded array of microwires. The glass can have a curved surface that conforms to the inner radius of the retina. The sensor-stimulator 108 can include a microelectronic imaging device that can be made of thin silicon containing integrated circuitry that convert the incident photons to an electronic charge.

An image processor 102 is in signal communication with the sensor-stimulator 108 via cable 104 which extends through surgical incision 106 through the eye wall (although in other embodiments, the image processor 102 is in wireless communication with the sensor-stimulator 108). In an exemplary embodiment, the image processor 102 is analogous to the sound processor/signal processors of the auditory prostheses detailed herein, and in this regard, any disclosure of the latter herein corresponds to a disclosure of the former in an alternate embodiment. The image processor 102 processes the input into the sensor-stimulator 108, and provides control signals back to the sensor-stimulator 108 so the device can provide processed and output to the optic nerve. That said, in an alternate embodiment, the processing is executed by a component proximate to or integrated with the sensor-stimulator 108. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 108 captures light/images, which sensor-stimulator is implanted in the recipient.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner.

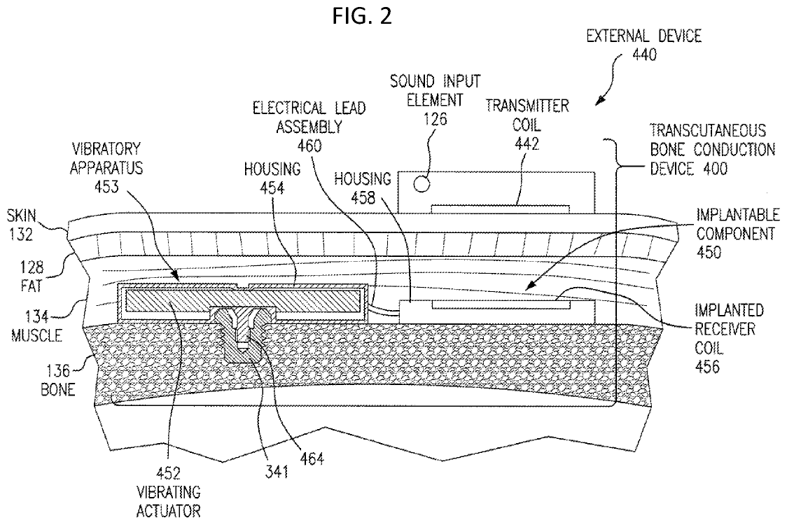

FIG. 2 depicts an exemplary embodiment of a transcutaneous bone conduction device 400 according to an embodiment that includes an external device 440 and an implantable component 450. The transcutaneous bone conduction device 400 of FIG. 2 is an active transcutaneous bone conduction device in that the vibrating electromagnetic actuator 452 is located in the implantable component 450. Specifically, a vibratory element in the form of vibrating electromagnetic actuator 452 is located in housing 454 of the implantable component 450. In an exemplary embodiment, much like the vibrating electromagnetic actuator 342 described above with respect to transcutaneous bone conduction device 300, the vibrating electromagnetic actuator 452 is a device that converts electrical signals into vibration.

External component 440 includes a sound input element 126 that converts sound into electrical signals. Specifically, the transcutaneous bone conduction device 400 provides these electrical signals to vibrating electromagnetic actuator 452, or to a sound processor (not shown) that processes the electrical signals, and then provides those processed signals to the implantable component 450 through the skin of the recipient via a magnetic inductance link. In this regard, a transmitter coil 442 of the external component 440 transmits these signals to implanted receiver coil 456 located in housing 458 of the implantable component 450. Components (not shown) in the housing 458, such as, for example, a signal generator or an implanted sound processor, then generate electrical signals to be delivered to vibrating electromagnetic actuator 452 via electrical lead assembly 460. The vibrating electromagnetic actuator 452 converts the electrical signals into vibrations.

The vibrating electromagnetic actuator 452 is mechanically coupled to the housing 454. Housing 454 and vibrating electromagnetic actuator 452 collectively form a vibratory apparatus 453. The housing 454 is substantially rigidly attached to bone fixture 341.

The implantable component 450 can include a battery or other power storage device, and can be rechargeable.

The embodiments of the implantable components above are examples, and at least some of the various components above can be the same as or correspond to proxies for other implantable devices, such as a middle ear implant or a DACS, etc. The actuator of the device of FIG. 2 can be a proxy for the actuator of a middle ear implant. The coil 456 can be a proxy for the coil of the DACS. Accordingly, any element disclosed herein with respect to one implant can be present in another implant or an analogous component can be therein providing that the art enables such unless otherwise identified.

The examples of implantable devices above are devices that are powered by and/or recharged by a transcutaneous inductance link. Power is transferred from the external component to the implanted component/implantable component via an inductance link. Embodiments include external components/portions thereof that generate an inductance field for powering and/or charging the implant, as will now be detailed.

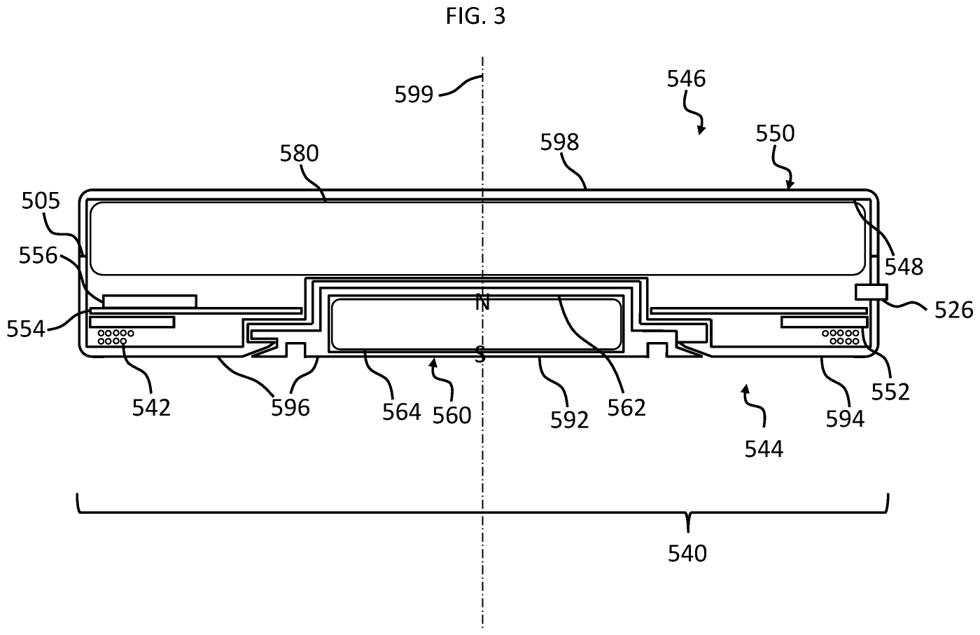

FIG. 3 depicts a cross-sectional view of an exemplary external component 540 corresponding to a device that can be used as external component 142 of FIG. 1 or for example external component 440 in the embodiment of FIG. 2, or any other external component usable with the various prostheses detailed herein. In an exemplary embodiment, external component 540 has all of the functionalities detailed above with respect to external component 142 or external component 440, etc.

External component 540 comprises a first subcomponent 550 and a second subcomponent 560. It is briefly noted that back lines have been eliminated in some cases for purposes of ease of illustration. It is further noted that unless otherwise stated, the components of FIG. 3 are rotationally symmetric about axis 599, although in other embodiments, such is not necessarily the case.

In an exemplary embodiment, external component 540 is a so called off the ear sound processor. In this regard, in the exemplary embodiment of FIG. 3, the external component 540 includes a sound capture apparatus 526, which can correspond to the sound capture apparatuses 126 detailed above, and also includes a sound processor apparatus 556 which is in signal communication with or located on or otherwise integrated into a printed circuit board 554. Further as can be seen in FIG. 3, an electromagnetic radiation interference shield 554 is interposed between the coil 542 and the PCB 554 and/or the sound processor 556. In an exemplary embodiment, the shield 552 is a ferrite shield. These components are housed in or otherwise supported by subcomponent 550. Subcomponent 550 further houses or otherwise supports RF coil 542. Coil 542 can correspond to the coil 442 detailed above. In an exemplary embodiment, sound captured by the sound capture apparatus 526 is provided to the sound processor 556, which converts the sound into a processed signal which is provided to the RF coil 542. In an exemplary embodiment, the RF coil 542 is an inductance coil. The inductance coil is energized by the signal provided from the processor 556. The energized coil produces an electro-magnetic field that is received by an implanted coil in the implantable component 450, which is utilized by the implanted component 450 as a basis to evoke a hearing percept as detailed above.

The external component 540 further includes a magnet 564 which is housed in subcomponent 560. Subcomponent 560 is removably replaceable to/from subcomponent 550. In the exemplary embodiment of FIG. 3 when utilized in conjunction with the embodiment of FIG. 3, the magnet 564 forms a transcutaneous magnetic link with a ferromagnetic material implanted in the recipient (such as a magnet that is part of the implantable component 450, etc.). This transcutaneous magnetic link holds the external component 540 against the skin of the recipient. In this regard, the external component 550 includes a skin interface side 544, which skin interface side is configured to interface with skin of a recipient, and an opposite side 546 that is opposite the skin interface side 544. That is, when the external component 540 is held against the skin of the recipient via the magnetic link, such as when the external component 540 is held against the skin overlying the mastoid bone where the implantable component is located in or otherwise attached to the mastoid bone, side 546 is what a viewer who is looking at the recipient wearing the external component 540 can see (i.e., in a scenario where the external component 540 is held against the skin over the mastoid bone, and a viewer is looking at the side of the recipient's head, side 546 would be what the viewer sees of the external component 540).

Still with reference to FIG. 3, skin interface side 544 includes skin interface surfaces 592 and 594. Skin interface surface 592 corresponds to the bottom most surface of the subcomponent 560, and skin interface surface 594 corresponds to the bottom most surface of the subcomponent 550. Collectively, these surfaces establish surface assembly 596. Surface assembly 596 corresponds to the skin interface surfaces of the external component 540. It is briefly noted that in some exemplary embodiments, the arrangement of the external component 540 is such that the subcomponent 560 can be placed into the subcomponent 550 such that the bottom surface 592 is recessed relative to the bottom surface 594, and thus the surface 592 may not necessarily contract or otherwise interface with the recipient. It is further briefly noted that in some alternate exemplary embodiments, the arrangement of the external component 540 is reversed, where surface 594 does not contact the recipient because surface 592 remains proud of surface 594 after insertion of the subcomponent 560 into the subcomponent 550.

It is briefly noted that as used herein, the subcomponent 550 is utilized to shorthand for the external component 540. That is, external component 540 exists irrespective of whether the subcomponent 560 is located in the subcomponent 550 or otherwise attached to subcomponent 550.

In the embodiment of FIG. 3 the external component 550 is configured such that the subcomponent 560, and thus the magnet 564 and the housing containing magnet 564 (housing 562), is installable into the external component 540 (i.e., from subcomponent 550) from the skin interface side 544, and thus is installable into the housing 548 at the skin interface side. Also, in some embodiments, the subcomponent 560 is removable from the external component 550. Still with reference to FIG. 3, it can be seen that the external component 540 includes a battery 580. In an exemplary embodiment, the battery 580 powers the sound processor 556 and/or the RF coil 542. As can be seen in FIG. 3, the battery 580 is positioned between the subcomponent 560, and thus the magnet 564, and the side 546 of the external component 540 opposite the side 544 configured to interface with the skin.

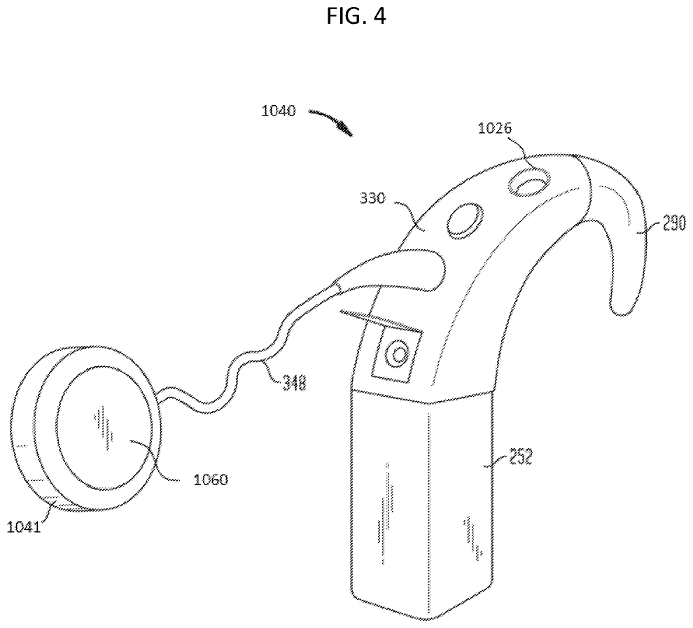

FIG. 4 depicts an alternate embodiment of an external component of an external device, BTE device 1040, which can be used in place of external components detailed above, and otherwise has the functionality thereof in at least some exemplary embodiments. More specifically, FIG. 4 depicts a perspective view of a BTE device of a hearing prosthesis. BTE device 1040 includes one or more microphones 1026, and may further include an audio signal under a cover 220 on the spine 330 of BTE device 1040. It is noted that in some other embodiments, one or both of these components (microphone 1026 and/or the jack) may be located on other positions of the BTE device 1040, such as, for example, the side of the spine 330 (as opposed to the back of the spine 330, as depicted in FIG. 4), the ear hook 290, etc. FIG. 4 further depicts battery 252, that is a rechargeable battery, and ear hook 290 removably attached to spine 330.

In an exemplary embodiment, the external component 1040 includes a sound processor or the like located in spine 330. The sound processor is in electronic communication with headpiece 1041 via cable 348. Headpiece 1041 can include an RF coil such as those detailed above. Concomitant with the teachings detailed above with respect to the sound processor of various other embodiments detailed herein, sound captured by the microphone 1026 is transduced into an electrical signal that is supplied to the sound processor, either directly or indirectly. The sound processor processes the signal and converts it into a signal or otherwise processes the signal so as to output a signal via cable 348 to the RF coil located in headpiece 1041, where the RF coil functions according to the teachings detailed above, in at least some exemplary embodiments.

Headpiece 1041 includes a magnet apparatus 351. This magnet apparatus can have the functionality of the subcomponent 550 detailed above.

While the embodiment depicted in FIG. 4 utilizes a cable 348 to establish communication between the spine 330 and the headpiece 1041, in an alternative embodiment, a wireless link is utilized to communicate between the spine 330 and the headpiece 1041.

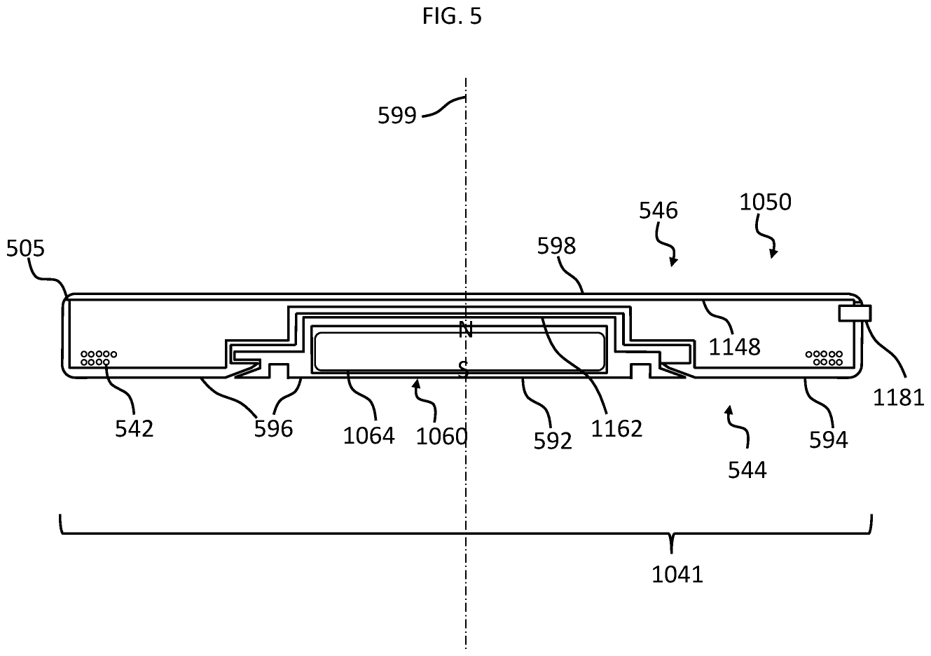

FIG. 5 depicts a cross-sectional view of the headpiece 1041. Here, FIG. 5 is presented with the same frame of reference with respect to FIG. 3 detailed above. Like reference numbers have been utilized in some instances for convenience of conveyance of concept. As can be seen, headpiece 1041 includes a subcomponent 1050 and a subcomponent 1060. In an exemplary embodiment, the subcomponent respectively corresponds, in a conceptual manner, to subcomponents 550 and 560 detailed above. In this regard, subcomponent 550 includes a housing 1148, which contains an RF coil 542. The housing 1148 comprises two sub housings that are joined together at seam 505. Subcomponent 1050 includes cable jack 1181, which is configured to connect the cable 348 to the headpiece 1041.

Subcomponent 1060 includes housing 1162 which contains magnet 1064. In an exemplary embodiment, the functionalities of the components depicted in FIG. 5 can correspond to the functionalities of similar components presented in FIG. 4. In this regard, some of these functionalities will be described in detail below. Briefly, it is noted that the embodiment of FIG. 5 is such that the housing 1148 has a height that is less than the housing 548 of the embodiment of FIG. 4. In the exemplary embodiment depicted in FIG. 5, there is no battery and no sound processor present in headpiece 1041 (because these components can be located in the spine 330, where headpiece 1041 is in signal communication with via cable jack 1181). Thus, the housing can be thinner.

In the embodiment of FIG. 5, the subcomponents interface with one another and are removable and/or attachable with respect to one another in a manner that is the same as or otherwise similar to the embodiment of FIG. 3, where again, additional details of such will be provided below.

In view of the embodiment of FIG. 5, it is to be understood that in an exemplary embodiment, there is a body piece, such as, for example, head piece 1041 (it is noted that in some alternate embodiments, the teachings detailed herein and/or variations thereof can be applicable to components that are not headpieces, but instead, or torso pieces and/or limb pieces etc.) configured for transcutaneous communication with a component implanted in a recipient (e.g., implantable component 450 of FIG. 3, or the implantable component of FIG. 1). In view of FIG. 5, it can be seen that the body piece includes an RF coil 542 and a magnet apparatus in the form of a subcomponent 1060. As can be seen, the RF coil is located on a first side of the body piece relative to an opposite side of the body piece. In this regard, with respect to a plane normal to longitudinal axis 599 bifurcating the geometric body established by the headpiece 1041 (a plane through the geometric center of the headpiece 1041), the RF coil 542 would be located entirely and/or a majority of the RF coil 542 would be located on one side of that plane. Here, the sides of the body piece can be side 544 and 546, the side being opposite to one another. It is further noted that in an exemplary embodiment, with respect to a plane normal to the longitudinal axis 599 bifurcating the center of mass established by the subcomponent 1050 (i.e., without subcomponent 1040 which, owing to the weight of the magnet 1064 would bias the center of mass to one side versus the other a disproportionate amount), the RF coil 542 would be located entirely and/or a majority of the RF coil 542 would be located on one side of the plane. That said, in an alternate embodiment, with respect to a plane normal to the longitudinal axis 599 bifurcating the center of mass established by the entire headpiece 1041 (and also, with respect to the embodiment of FIG. 4 (where external component 550 also corresponds to a body piece), a plane bifurcating center of mass established by the entire external component 540), the RF coil 542 would be located entirely and/or a majority of the RF coil 542 would be located on one side of this plane.

Consistent with the teachings associated with FIG. 3, the embodiment of FIG. 5 is such that the aforementioned first side is a skin interface side (side 544) that consists of a first structure and a second structure. Here, the first structure can correspond to the bottom subcomponent of the housing 1148 and/or 548 (e.g., with respect to the embodiment of FIG. 4, subcomponent 547, which establishes surface 594). Still further, the second structure can be established by the magnet apparatus 1060 (or 560), where the bottom of housing 1162 (corresponding to housing 562 of the magnet apparatus 560) of magnet apparatus 1060 establishes surface 592. In this exemplary embodiment, the first structure established by the housing 1148 houses or otherwise contains the RF coil 542, and the second structure established by housing 1162 houses or otherwise contains the magnet 1064.

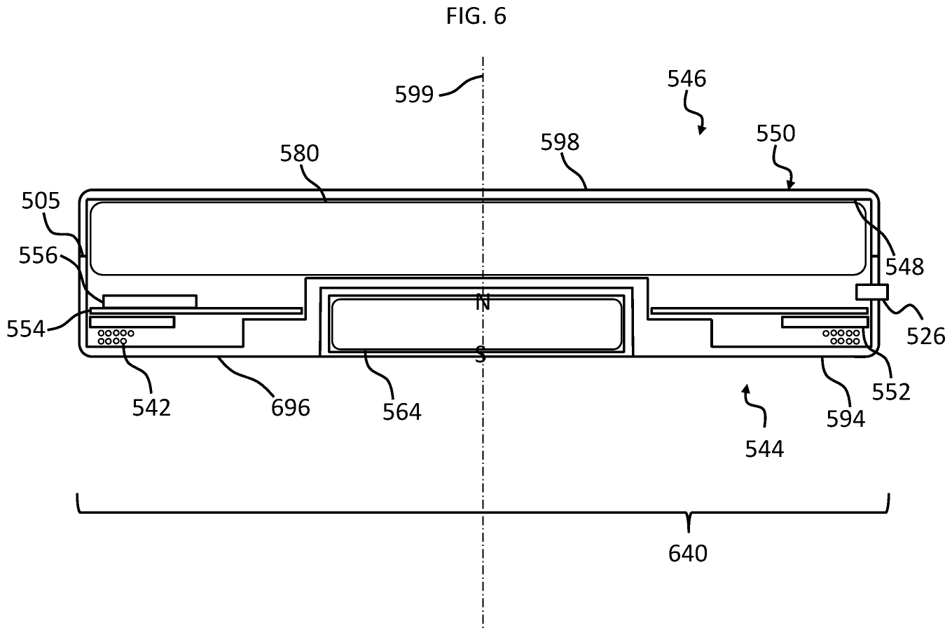

FIG. 6 depicts another exemplary embodiment of an external component 640 that corresponds to the external component 540 above, except that the magnet apparatus is not removable, and in an alternative embodiment, the magnet apparatus is removed from the opposite side 546 (the battery could be a doughnut battery, to enable movement of the magnet apparatus therethrough, or the external component can be configured so that the battery is removable to access the magnet apparatus) as opposed to from the skin interface side 544. This results in a skin interface surface 696 that is without seams and is otherwise uniform and unbroken from side of the external component to the other with respect to the skin interface side 544. In an exemplary embodiment, any one or more of the features of the embodiment of FIG. 6 can be present in the headpiece of the embodiment of FIG. 5 detailed above. In this regard, the skin interface side 544 of the headpiece 1050 can be without seams and otherwise unbroken as is the case with the embodiment of FIG. 5.

The embodiments of FIGS. 1 to 6 are devices that transfer power (and in some embodiments, data, control data) in some form or another to an implanted device. In some instances, such as the embodiments where the implant is a totally implantable prosthesis, such as a totally implantable hearing prosthesis, the implanted portion includes some form of power storage device, such as a battery, that is rechargeable. The external device can be utilized to charge/recharge that battery utilizing the inductance link from the external component of the implantable component. In this regard, any of the embodiments of FIGS. 1 to 6 can correspond to a generic implant charger in that the external component does not have the sound processor or one or more of the other features detailed above, and instead is directed to solely recharging the implant. The external component can be characterized in at least some exemplary embodiments, as a battery (whether rechargeable or disposable), and an inductance coil that is part of an inductance communication system that is configured to generate an inductance field that can communicate with the implant/transfer power to the implant, and some form of circuitry, which can include logic circuit or control circuit, such as an inductance coil drive circuitry. Some embodiments can include more functional components unrelated to such, but other embodiments can be limited to exactly that (there could be an on-off switch in some such embodiments, and other components, such as a recharge switch (to enable recharging of the battery the external component in some embodiments—in other embodiments, there could be logic that detects when the battery is to be recharged, and thus there may not necessarily be a dedicated recharge switch) but such would be related to the functionality of recharging the implant/transferring power to the implant so that the implant can be recharged—this as opposed to a volume control, or a microphone, which are unrelated to the functionality of recharging the implant or recharging the battery of the external component so that it can be utilized to recharge the implant).

The above said, in some other embodiments, the external component is a device that controls or otherwise provides data (as opposed to simply power) to the implant. It is noted that providing data is not mutually exclusive with providing power. In this regard, in the exemplary embodiment of an external component of a partially implantable hearing prostheses detailed above, such as a partially implantable cochlear implant (non-totally implantable hearing prosthesis), the external component provides power and data/power and control signals that are received by the implant and for all intents and purposes immediately utilized to provide stimulus to the recipient (e.g., to power the cochlear electrode array to provide current to the tissue of the recipient, which current is applied in a controlled manner to evoke the desired hearing percept).

The teachings herein are utilized in at least some embodiments with respect to both types of an external component—the limited external charger and the broader external data source device (which can include the external devices detailed herein that include a sound processor, but also can include by way of example only and not by way of limitation a device that has an external sound processor and a device that simply has microphones or other sound capture devices or other data capture devices, which then provide a signal based there on to the implant, where the implant processes that signal, etc., to evoke the desired hearing percept). It is also noted that the functionality of the two types of external components are not mutually exclusive—an external device can have the functionality of the external sound processors detailed herein, but also can have the functionality of recharging an implanted power storage device.

Transcutaneous power transfer from the external component to the implantable component, such as during recharging of the implantable prosthesis (and thus recharging of the implantable/implanted power storage device—any disclosure herein of recharging implantable prostheses corresponds to a disclosure of recharging the implantable/implanted battery, and vice versa) can result in an increase in temperature of at least some portions of the external component relative to that which would otherwise be the case (e.g., there could be an increase in temperature because the external component is in the sun for example, or because it is hot outside and the recipient just recently moved from inside an air-conditioned environment to an environment that is not air-conditioned (e.g., outside, a factory floor, a warehouse, etc.). In some scenarios, this increase in temperature can be well within comfort and/or safety levels, but in other scenarios, this may not necessarily be the case. The temperature increase can result in a temperature of a skin interface surface, such as skin interface surface 594 and/or skin interface surface 592 and/or the surface assembly 596 and/or the skin interface surface 690, to increase to a level that is uncomfortable and/or unsafe. Hereinafter, these surfaces will be referred to herein as the skin interface surface for linguistic economy. Any reference to such corresponds to a reference to one or more of the aforementioned surfaces unless otherwise noted.

Teachings herein can prevent excessive heating of the external component and/or of the skin interface surface so that the device meets the requirements/guidelines of EN60601-1: “Protection against excessive temperatures and others hazards” which includes some temperature limitation tables applicable to medical equipment that is operated in worst-case normal use including the ambient operating temperature specified in the technical description and/or ISO14708-1/-7, which details that no outer surface of an implantable part of the active implantable medical device shall be greater than 2° C. above the normal surrounding body temperature of 37° C. when implanted, and when the active implantable medical device is in normal operation or in any single-fault condition and/or ISO 14708-3, which details that physical temperature-time limits on heating tissue is given by CEM43, where the temperature of the implanted metal must stay below 43° C.

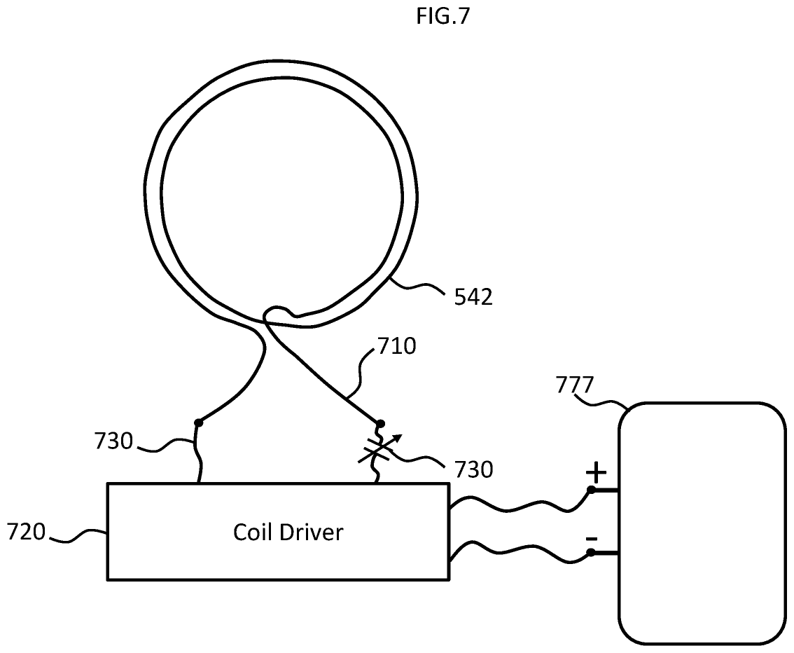

FIG. 7 depicts a high level functional schematic of an inductance recharging system and/or inductance communication system that generates and inductance field to charge and/or otherwise power an implantable component, along with a DC battery 777. The inductance coil 542 can correspond to any of the inductance coil detailed herein, and as can be seen, the coil includes lead portions 710 which are linked to leads 730 of a coil driver 720. In an exemplary embodiment, this coil driver induces an alternating current in the coil 542, and with coil tuning apparatus 730, an inductance field is thus generated and is utilizable to recharge or otherwise power the implant via an inductance link there with. In this regard, FIG. 7 depicts the functional schematic of components of the external devices detailed herein and/or variations thereof.

The coil driver 720 includes circuitry configured to convert DC power from the battery 777 into an alternating current (e.g., by using switching diodes, etc.) that is then applied to the coil 542 to generate the inductance field. The coil driver can include circuitry to vary the inductance field or otherwise vary the amount of current flowing through the coil 542 and/or very the voltage flowing through coil 542 so as to vary or otherwise control an amount of power that is transferred from the external component to the implant (e.g., to reduce a recharge time). In an exemplary embodiment, the driver is a power conversion unit, converting DC current to AC current can utilize one or two or more push-pull switches/transistors. In some embodiments, two half bridges are used to establish a full bridge driver and allow a full AC conversion. This full bridge can be driven by a controller (circuitry configured to do so) which can ensure different switches used are synchronized altogether so that energy wastage is minimized (including prevented). Such device is used for instance on motor drivers, but more recently on wireless chargers.

In an exemplary embodiment, battery 777 corresponds to any one of the batteries detailed above with respect to the external components. The battery 777 can be rechargeable or can be a disposable battery. In an exemplary embodiment, the arrangement of FIG. 7 is embodied in a unitary dedicated external charging device in the form of an off the ear device, such as the device of FIG. 3 or the device of FIG. 6. In an exemplary embodiment, the arrangement of FIG. 7 is embodied in a unitary dedicated external charging device in the form of a BTE device with a headpiece, such as the device of FIG. 4, where battery 252 corresponds to battery 777, and the coil driver is located in the spine 330, and the coils are located in the headpiece 1041. Still, in other embodiments, the arrangement of FIG. 7 can be embodied or otherwise combined with an external sound processor or the like.

Temperature heating of the external component can be a result of a coil and/or driver and/or the battery discharge. Teachings herein can utilize technologies to mitigate the heating effects.

Embodiments can include the utilization of heat pipes, such as ultra-thin and/or flat heat pipes, and/or small thermoelectric coolers (TEC) and miniature fans. Embodiments utilize heat pipes in some instances to extract heat from one side of the external component, such as the skin interface side, and transmit/convey the heat to the other side, thus cooling the one side relative to that which would be the case in the absence of such heat transfer.

An exemplary embodiment utilizes a heat pipe, such as a flat heat pipe, as a charging coil 542. In an exemplary embodiment, the coils can be established by a hose that is made of pure copper or a copper alloy or any other appropriate material that is suitable for utilization in a high-quality inductance coil. This can enable the extraction of heat where it is produced/generated. Indeed, for an implantable device, the heating typically occurs around the implant and the charging coil on the skin. In some embodiments, the coil is less than or equal to 0.25, 0.3, 0.35, 0.4, 0.45, 0.5, 0.6, 0.7, 0.8, 0.9, 1.0, 1.25, 1.5, 1.75, 2, 2.5, 3, 3.5, 4, 4.5, 5, 5.5, 6, 6.5, 7, 8, 9, or 10 mm, or any value or range of values therebetween in 0.01 increments from the skin interface surface, and a distance between the external coil and the implanted coil can be less than or equal to 5, 6, 7, 8, 9, 10, 11, 12, 13, 14, 15, 16, 17, 18, 19, 20, 21, 22, 23, 24, 25, 26, 27, 28, 29 or 30 mm or any value or range of values therebetween in 0.1 mm increments. Extracting the heat with the part that transmits the power to the implant can be utilitarian to ensuring or otherwise enabling fast charging, or at least charging within a tolerably short amount of time, because such can mitigate overheating in some embodiments. In an exemplary embodiment, a height of the heat pipe can be less than or equal to 0.5, 0.6, 0.7, 0.8, 0.9, 1.0, 1.1, 1.2, 1.3, 1.4, 1.5, 1.6 or 1.7 mm or any value or range values therebetween in 0.1 mm increments, and width can be less than or equal to 0.5, 0.6, 0.7, 0.8, 0.9, 1.0, 1.1, 1.2, 1.3, 1.4, 1.5, 1.6 or 1.7 mm or any value or range values therebetween in 0.1 mm increments. In an exemplary embodiment might be a Cooler Master Slim heat pipe, or a Petra-flex heat pipe.

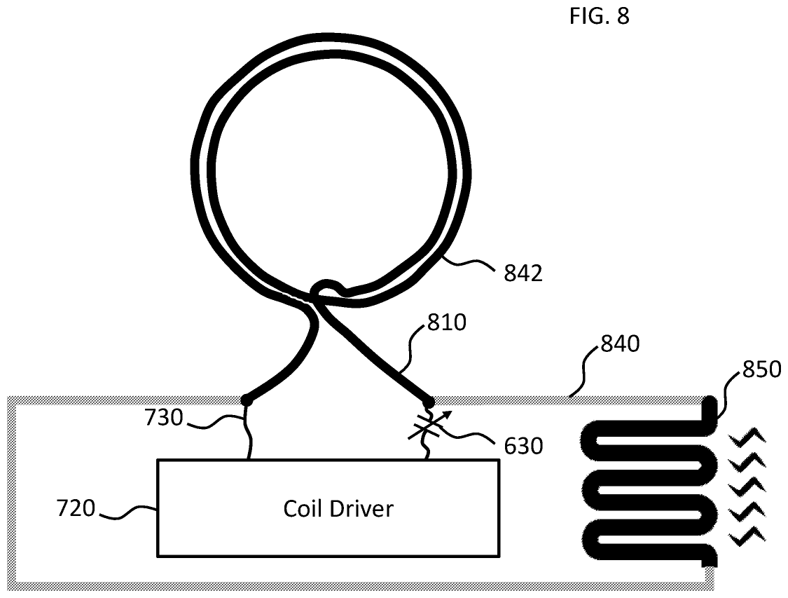

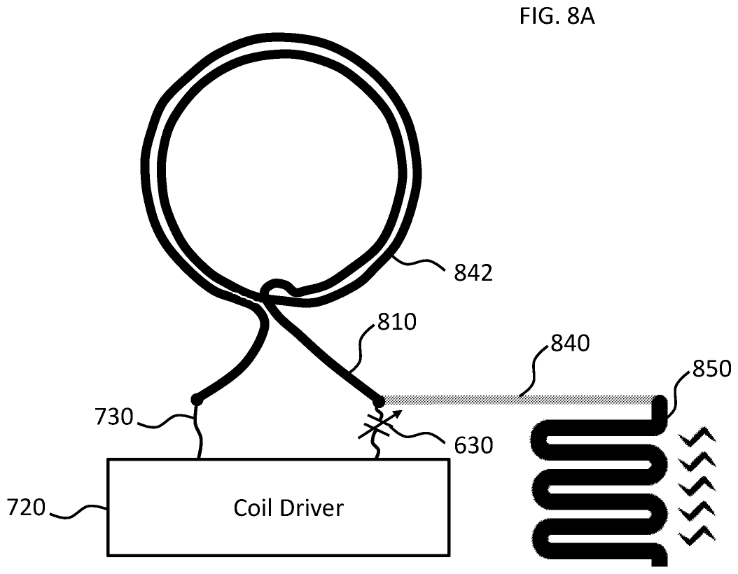

FIG. 8 depicts an exemplary embodiment that utilizes a heat pipe, and in this regard, a flattened heat pipe, as the coil 842 in the leads 810. In this exemplary embodiment, the heat pipe is an electrically conductive heat pipe with respect to the coil portions and the leads portions. The coil driver is electrically connected to the heat pipe via electrical leads 730. Piping 840 is fluidically connected to the leads 810 so as to enable fluid flow from the coil 842 and the leads 810 to the radiator 850 (more on this in a moment). The piping 840 in this exemplary embodiment is electrically nonconductive, thus enabling the coil 842 to be electrically decoupled from the rest of the heat transfer system. An exemplary embodiment, this is not the case—the piping and/or the radiator can also be electrically conductive. In some embodiments, the section of 840 can be a heat pipe in some embodiments, and in other embodiments, it is simply piping to transfer fluid, and thus transfer heat via mass transfer.

The above said, in some embodiments, a portion of the piping can be conductive. Indeed, the heat conduction side of the system can be grounded. By way of example, the radiator 850 can be a ground plane for the inductance system. In an exemplary embodiment, the piping between element 810 and the radiator 850 can be conductive. In an exemplary embodiment, the piping can be the ground plane.

An exemplary embodiment, a tuner cap/tuning cap or the like that is utilized to tune the system is placed “between” (relative to electrical and/or heat transfer paths) the coil 842 and the area where heat is transferred from the coil/the path from a tuning device attaches between the coil and the area where heat is transferred to from the coil. This as opposed to placing a tuner at the other lead 730 of the embodiment of FIG. 8 relative to that which is shown in FIG. 8.

It is briefly noted that while the arrangement shown in FIG. 8 depicts a circuit for fluid to flow around, in an alternate embodiment, there is no true circuit, at least with respect to the view of FIG. 8. In an exemplary embodiment, the fluid might flow back and forth within the heat pipe. By rough analogy, from a distance, a Lionel train on a train platform would travel in a circuit akin to that of FIG. 8 (say a circular train track), whereas a ski lift chair, when viewed from a distance, will by analogy follow the just described “back and forth” path. FIG. 8A depicts such a system.

In any event, in an exemplary embodiment, heat transfer fluid can flow through the piping 840 into the heat pipes and transfer heat generated by the inductance coil into the fluid, and then the fluid can be transported to the radiator 850 where the heat is then radiated out of the system, and then the fluid continues to flow through the circuit back into the coil and the process is repeated.

The radiator 850 can be any type of radiator that can enable the teachings detailed herein. In an exemplary embodiment, a fan with the like can be cold located with or otherwise in communication with the outer surface of the radiator to enhance heat transfer there from. In an exemplary embodiment, the radiator can be a thermoelectric cooler and/or can be in conductive heat transfer communication with a thermoelectric cooler. In an exemplary embodiment, a DC current can be harnessed from the battery 777 directly or indirectly and/or can be obtained from another power source, which DC current can be utilized to achieve a Peltier effect, and thus bring heat from one side of the device to the other.

In another exemplary embodiment, a heatsink can be placed in conductive heat transfer communication with the radiator and/or the radiator can be an expanded heat sink relative to the heat pipe. Any device, system, and/or method that can enhance heat extraction from the heat pipe/fluid flow path can be utilized in at least some exemplary embodiments.

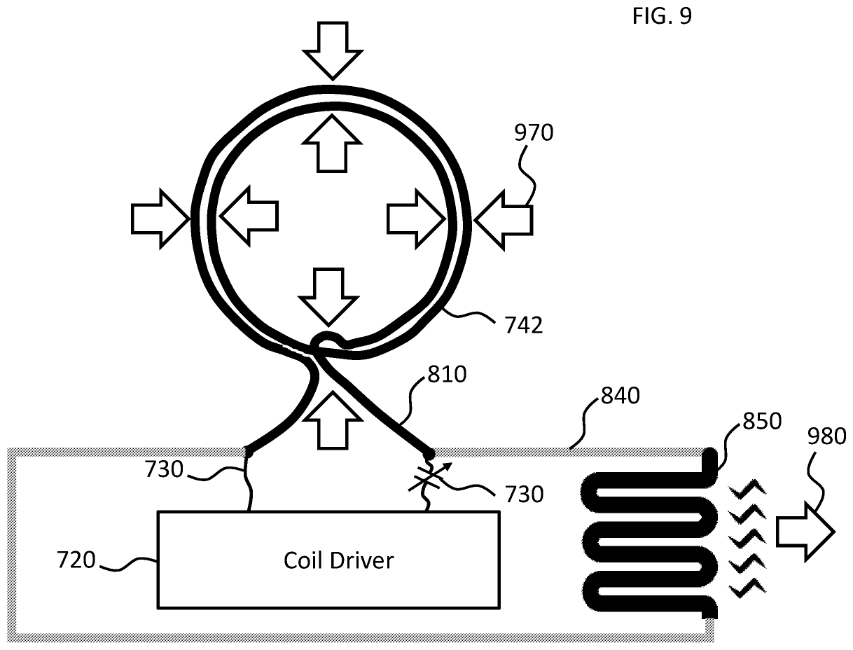

FIG. 9 presents a schematic depicting heat flow into the coil 742, represented by arrows 970 (eight arrows pointing to the coil 742). FIG. 9 also presents a schematic depicting heat flow out of the radiator 850, represented by arrow 980.

In an exemplary embodiment, the coils of the embodiments of FIGS. 1-6 are utilized with the coil of the embodiment of FIG. 8 and/or the arrangement of FIG. 8 is used with the embodiments of FIGS. 1-6. The coil driver can correspond to the circuitry, at least in part, of those embodiments.

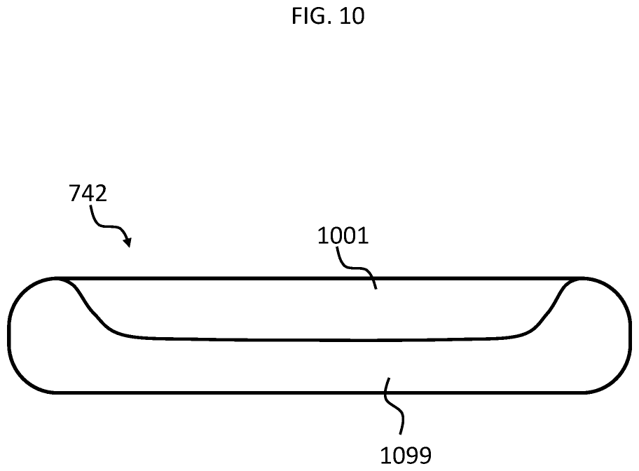

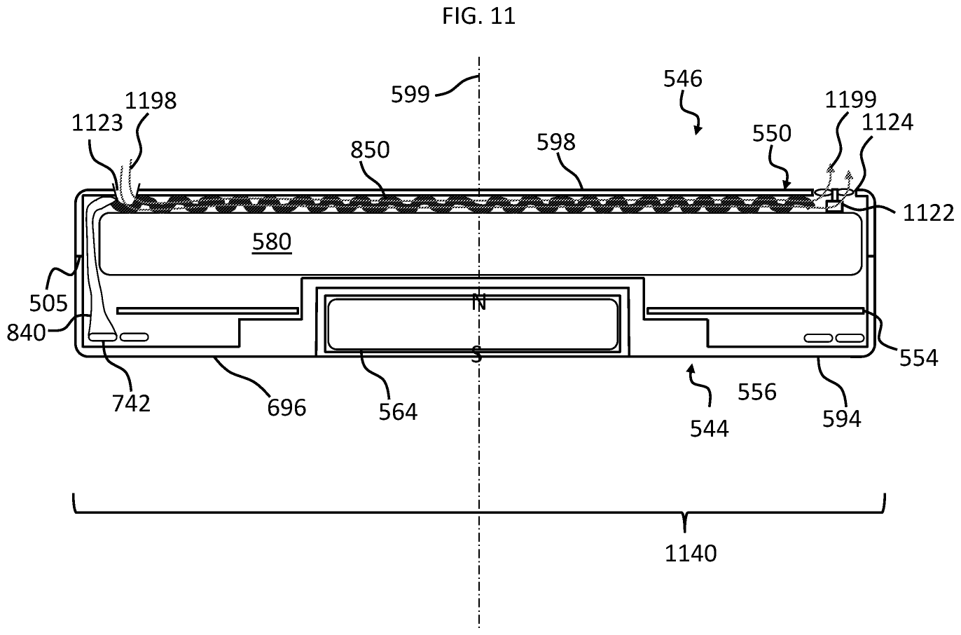

FIG. 10 presents a cross-section of an exemplary flat heat pipe 742. Here, there are two channels, a vapor channel 1001 and a liquid flow channel 1099. These channels are utilized to transfer heat from the area proximate the skin of the recipient to a location elsewhere at the external component. In this regard, FIG. 11 depicts an exemplary external component 1140 that parallels the arrangement of FIG. 6 above. Here, it can be seen that the inductance coil 742 are in the form of heat pipes. In this embodiment, the coil 542 of the embodiment of FIG. 6 has been replaced with the coil 742 that is in the form of a heat pipe. Also seen in FIG. 11 is the piping 840 that is not electrically conductive as detailed above. Piping 840 is depicted as transforming from a flat arrangement to a circular arrangement with distance from the plane of the coil. That said, in an exemplary embodiment, the arrangement of a flat heat pipe can be present with respect to the piping 840. As seen, the piping 840 extends to the radiator 850, which is located away from the skin interface side 544. In this embodiment, the radiator is above the battery 850 but in other embodiments, this can be located on the side of the battery, etc. Any arrangement that will enable the teachings detailed herein can be utilized in at least some exemplary embodiments. As seen, convective airflow is also utilized to enhance heat transfer. Here, there is an intake vent 1123 and an outlet vent 1124. A fan 1122 is located in or proximate the outlet, but that can be located in/proximate the inlet and/or two or more fans can be utilized, one at the intake and one at the outlet. Moreover, the fan can be positioned elsewhere. Any device, system, and/or method that can enable airflow 1198 across the radiator 850 to enhance heat transfer can be utilized in at least some exemplary embodiments. In this embodiment, the fan is powered by the battery 580. In another exemplary embodiment, the fan could be powered by a solar cell or the like. In an exemplary embodiment, a miniature fan can be utilized such as a fan that has a casing that is 15×15×4 mm, which utilizes a current source that is between 2 and 3 Volts (e.g., 2.4 or 2.5 or 2.6 Volts).

Moreover, at least some embodiments do not necessarily utilize an electrically powered fan. In an exemplary embodiment, a manually operated system can be utilized to create a pressure difference to pull or push air across the radiator. By way of example only and not by way of limitation, a diaphragm arrangement can be utilized that will enable a recipient to utilize his or her finger to repeatedly deform the diaphragm and thus create a pressure difference to create airflow across the radiator 850. In this regard, this device can be considered a manual air pump actuatable by a finger. The diaphragm could extend over the radiator between the intake and the outlet—actually, this arrangement would result in both of those components being an intake and an outlet in an alternating manner as the diaphragm is pushed down and then released—pushing down would expel air out of the inside of the housing and thus through the intake an outlet, thus causing them both to be outlets—releasing the diaphragm and having the diaphragm spring back up to its at rest position would then create a lower pressure inside the housing which would then draw air in through the intake and the outlet.

It is noted that in some embodiments, an electric motor or some other device can be utilized to induce flow within the channels of the heat pipes/increase a flow rate within the channels beyond that which would otherwise be the case.

In view of the above, embodiments include a device, such as an external component of a prosthesis (whether a charging device or an integral component of the prosthesis), comprising an inductive power transmission apparatus, wherein the device includes a dedicated heat transfer arrangement configured to transfer away from the device heat that is generated when transferring power using the device. By “dedicated heat transfer arrangement,” it is meant that there is a recognizable structure in or on the device that one of ordinary skill in the art would recognize is there for the purposes of heat transfer. This as opposed to structure that exists because the device exists, which structure inherently transfers heat. In an exemplary embodiment, the inductance power transmission apparatus is configured to transmit inductance power to a person.

As noted above, in at least some exemplary embodiments, the external component can be a dedicated charger, while in other embodiments, the external component can be a data transmission device in addition to having the ability to transfer power to the implanted component. Thus, in an exemplary embodiment, the inductive power transmission apparatus can be an inductive communication apparatus.

Also, with reference to the embodiments detailed above that are configured to utilize airflow to enhance heat transfer, in an exemplary embodiment, the device is configured to induce movement of air through the device beyond that which would occur as a result of normal convention to enhance heat transfer from the dedicated heat transfer arrangement.

Consistent with the teachings above, in an exemplary embodiment, there is a device, such as an external component of a prosthesis, which device includes an inductive power transmission sub-system configured to transfer power to an implanted medical device, and a skin interface surface. The device further includes a cooling sub-system configured to cool the skin interface surface. Still further consistent with the teachings detailed above, in some embodiments, the cooling sub-system is integrated with the inductive power transmission sub-system. In this regard, as seen above, in some embodiments, the device is configured to transfer heat with a part that also transmits power, thereby cooling the skin interface surface. Conversely, also consistent with the teachings detailed above, in some embodiments, the cooling sub-system is not integrated with the inductive power transmission subsystem.

In some instances, the device is an off the ear charging device (for example, a device that does not include the sound processor components) and the cooling sub-system is configured to transfer heat from the skin interface.

In an exemplary embodiment, any of the devices of FIGS. 3, 5, 6, 11, etc., can represent an off the ear implant recharger if the sound processor, etc., is removed, if such is present. Note that an off the ear sound processor can also function as an implant recharger. The phrase implant recharger means that it is a dedicated recharger that has no other functionality.

In some embodiments, the device is a behind-the-ear (BTE) device and the skin interface is at a headpiece of the BTE device. In this regard, it is noted that behind the ear devices can be dedicated chargers, where the ear is utilized to support the battery and other components instead of utilizing the transcutaneous magnetic link to support the battery. This can, in at least some embodiments, enable the use of a larger and/or heavier battery relative to that which would otherwise be the case. In this embodiment, the BTE device simply has the sole functionality of charging the implant. Still, in other embodiments, the BTE device can be a sound processor/have the functionality of a sound processor. In both arrangements, there can be utilitarian value with respect to utilizing a cooling subsystem. With respect to embodiments that utilize a BTE device, the heat pipes can extend from the headpiece to the spine of the BTE device. The heat pipes can extend through cable 348, and a heat exchanger can be located in the spine 330. The heat pipes can enable the flow of cooling fluid from the headpiece to the heat exchanger and then back to the headpiece in a manner analogous to the operations detailed above. In an alternate embodiment, the cable 348 can be a heat exchange device. The cable could be ribbed or can include ribbed sections that would enhance heat transfer radiation and/or convection beyond that which would be the case with respect to a cylindrically shaped smooth cable.

It is also noted that in some embodiments, there can be a scenario where the body proper (spine, battery and/or ear hook) of the BTE device could experience a higher than desired temperature. In this regard, embodiments include a BTE device where the heat transfer arrangements and/or cooling arrangements herein are implemented in the BTE device body and/or are otherwise utilized to reduce a temperature of a surface of the BTE device body that contact skin relative to that which would otherwise be the case without such implementations. By way of example, the battery 252 could become heated during discharge (or charging—more on this in a moment) and/or the coil driver could produce heat, which coil driver is located in the spine 330, or any other component located in the BTE device body could produce heat, and thus there could be utilitarian value with respect to cooling the skin interfacing services. In an exemplary embodiment, heat pipes can be located proximate the outer surface of the battery 252 and/or the outer surface of the spine 330 and/or the outer surface of the ear of 290, which outer surface contacts the skin during normal use.

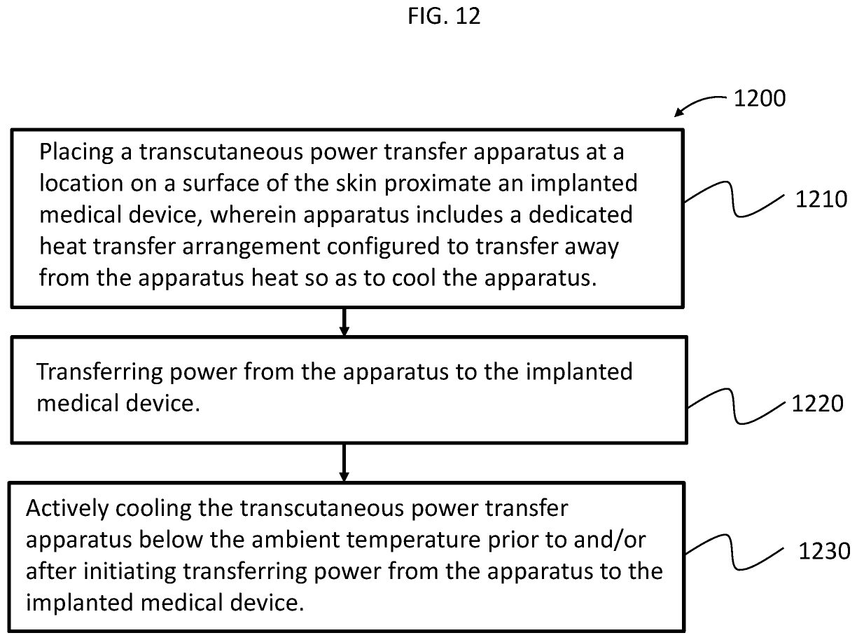

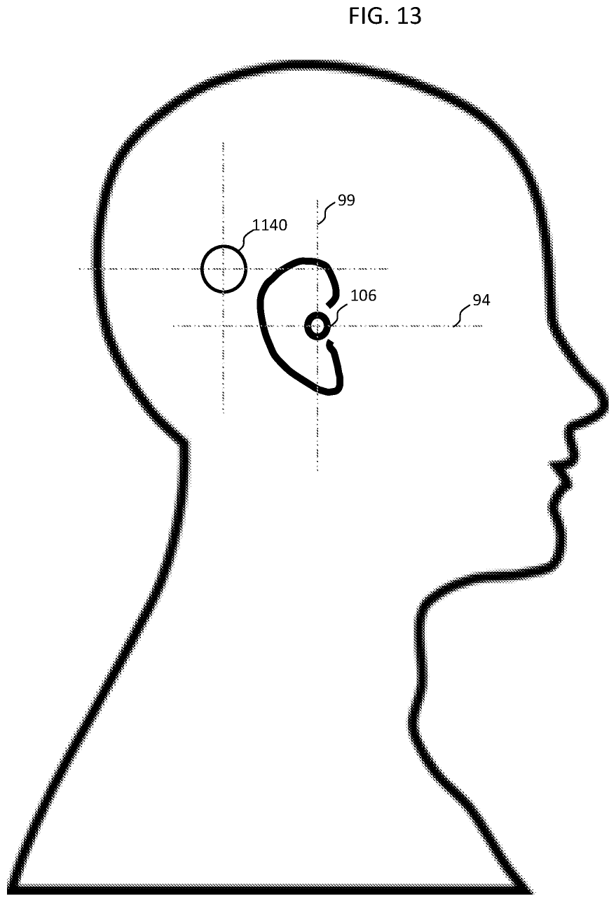

Embodiments also include methods. For example, FIG. 12 presents an exemplary flowchart for an exemplary method, method 1200. Method 1200 includes method action 1210, which includes placing a transcutaneous power transfer apparatus (e.g., the external components detailed herein, whether dedicated power recharging device or a data communication device that also transfers power) at a location on a surface of the skin proximate an implanted medical device, wherein apparatus includes a dedicated heat transfer arrangement configured to transfer away from the apparatus heat so as to cool the apparatus. With respect to the placement, this can be at a location off an ear of the recipient. This can correspond to placing the headpiece (e.g., 1140) at the location shown in FIG. 13, which should be considered to scale vis-à-vis a human factors engineering 50th percentile male or female of 40 years of age born in the United States of America as of Jan. 19, 2021. FIG. 13 depicts an exemplary placement of the external component 1140 against the head of a recipient from the frame of reference of the viewer looking at a right side of a recipient, where the recipient is looking ahead (the “right side” being the recipient's right side—the side of the recipient's right hand. Shown in FIG. 13 for purposes of reference is the pinna of the recipient, and the ear canal of the recipient 106. Horizontal axis 94 and vertical axis 99 are centered at the center of the outer opening of the ear canal 106. Horizontal axis 94 corresponds to the gravitational horizon, and vertical axis 99 is parallel to the direction of gravity. A distance in the X axis and/or the Y axis from the center locations of the canal 106 and the external component 140 can be any of 2, 2.25, 2.5, 2.75, 3, 3.25, 3.5, 3.75, 4, 4.25, 4.5, 4.75, or 5 inches or more, or any value or range of values therebetween in 0.01 inch increments.

In the embodiment discussed, the action of placing the headpiece against the skin of the recipient results in an inductance coil of the headpiece being effectively centered with an implanted inductance coil implanted underneath the skin of the recipient. The separation between the two coils can be less than, greater than or equal to 2, 3, 4, 5, 6, 7, 8, 9, 10, 11, 12, 13, 14, 15, 16, 17, 18, 19, or 20 mm or more, or any value or range of values therebetween in 0.1 mm increments.

Method 1200 further includes method action 1220 which includes transferring power from the apparatus to the implanted medical device. This can be done for recharging purposes, and can be done for powering the device in real time, or both. In this embodiment, this is done via the inductance link established by the external component and the implanted component. This can correspond to transferring only power to the implanted device or the transfer of both power and data to the implanted device, the latter being what happens in a partially implantable cochlear implant, for example.

But briefly, there can be utilitarian value in reducing a temperature of the external component prior to utilization relative to that which would otherwise be the case in the absence of affirmatively cooling the apparatus so that the total heat energy released will ultimately result in a lower overall temperature of the skin interface surface relative to that which would otherwise be the case, all other things being equal. In some embodiments, this is done using the on-board cooling devices as disclosed above, and in other embodiments, this is done by passive cooling but in a manner that can effectively achieve an analogous utilitarian result to that which is achieved utilizing the onboard cooling subsystems detailed herein. More on this below, but first, we continue to focus on utilization of the onboard cooling device.

Method 1200 thus also includes method action 1230, which includes actively cooling (as distinguished from mere cooling that results from mere natural heat transfer to the ambient environment (e.g., once recharging stops, heat generation ceases and the device will cool if heated above the ambient)) the transcutaneous power transfer apparatus/external component below the ambient temperature prior to and/or after commencing transferring power from the apparatus/external component to the implanted medical device. This as opposed to transferring heat away from the location at the time of commencement of transferring power from the apparatus to the medical device, and, in some embodiments, at any time during transferring power. Method action 1430 can be enabled utilizing the teachings detailed above, such as for example, utilizing a cooling subsystem, such as one of those detailed above by way of example.

And note that in some embodiments, method 1200 is executed without using per se the onboard cooling subsystem. That is, for example, method action 1200 can instead be executed where there is no dedicated heat transfer arrangement with the apparatus. This can be done by using the chilling/cooling apparatuses detailed below, for example, and the associated methods.

In an exemplary embodiment, the action of cooling is executed by moving a fluid from a location inside the apparatus and proximate a surface of the apparatus that interfaces with the surface of the skin to a location away from that location inside the apparatus. This can be achieved by way of example utilizing the pipes of the embodiment of FIG. 11 detailed above. It is noted that the movements of the fluid within the pipes can be a result of convection currents and/or can be induced via the utilization of a device that creates a pressure differential within the heat pipes. It is noted that fins or other heat transfer enhancing devices can be located on the inside surface of the wall that establishes the skin interface surface 690 to enhance the cooling.

Consistent with the teachings above, in an exemplary embodiment, method action 1230 is executed using thermoelectric cooling.

It is also noted that air can be blown across the hot sides of a Peltier device of an apparatus using such so as to transfer heat from those devices, thus cooling the apparatus. Of course, method action 1230 can be executed using heat pipe(s).

Thus, in an exemplary embodiment, the action of cooling the transcutaneous power transfer apparatus below the ambient temperature is executed using an onboard, relative to the apparatus, cooling sub-system.

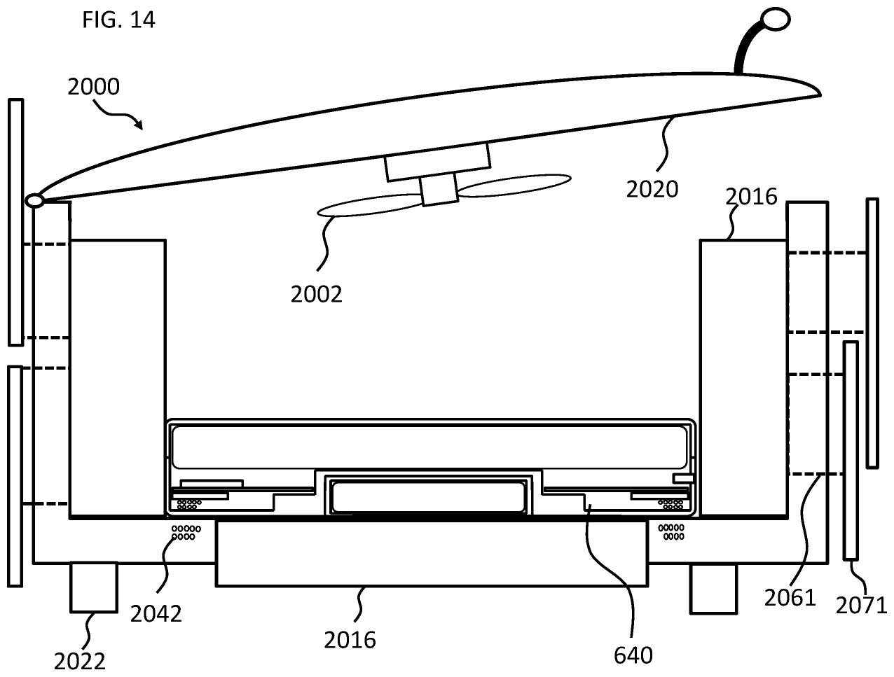

In an exemplary embodiment, the action of cooling the transcutaneous power transfer apparatus below the ambient temperature, method action 1230, is executed prior to commencement of transferring power from the apparatus to the implanted medical device, and in some embodiments, prior to transferring any power. By way of example only and not by way of limitation, this can be executed utilizing the onboard subsystems for cooling, or any other device that can be utilized to enable the teachings detailed herein. Doing this before commencement of recharging/before any recharging (and thus the numerical values/presentation of the method actions are not the exact order of execution), can have utilitarian value by powering the onboard cooling subsystem using power other than the battery of the external component (e.g., directly from the inductance coil received while recharging, or by a separate wired connection or by another inductance coil, or can be drawn from the battery of the external component while the battery of the external component is being recharged, thus extending the length of time to charge the battery). That said, in embodiments where an onboard cooling subsystem is not used or is not present (the modification of method action 1210), the device of FIG. 14 (more on this below), device 2000, is used to execute method action 1230.

In an exemplary embodiment, the action of cooling is not executed while the transfer of power from the apparatus to the implanted medical devices takes place. In an exemplary embodiment, no active cooling takes place while the external component is charging or otherwise recharging the implanted medical device.

In an exemplary embodiment, the action of active cooling ceases by a time period and/or no active cooling takes place during a time period that is more than or equal to 0.5, 0.75, 1, 1.25, 1.5, 1.75, 2, 2.25, 2.5, 2.75, 3, 3.5, 4, 4.5, 5, 5.5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, or 60 minutes or more, or any value or range of values therebetween in 0.1 minute increments (e.g., 3.2, 26.6, 5.3 to 55.5 minutes, etc.) prior to the commencement of transferring power from the apparatus to the implanted medical device (these times are measured from the point of commencement of the power transfer). In an exemplary embodiment, instead of the aforementioned times being measured from the commencement of transferring power, the aforementioned times are measured from the point in time where an amount of power has been transferred is that which results in at least more than and/or equal to an elevation of a state of charge of the implanted battery by at least and/or equal to 5, 10, 15, 20, 25, 30 or 35% of an implanted power storage device, such as a battery, during an action of transferring power from the apparatus to the implanted medical device.

Converse to the embodiment just described, in an exemplary embodiment, the action of cooling the transcutaneous power transfer apparatus below the ambient temperature (method action 1230) is executed after transferring power from the apparatus to the implanted medical device/after halting the transferring power. In an exemplary embodiment, the aforementioned temporal periods are measured from the point of cessation of the action of transferring power from the apparatus to the implanted medical device.

In an exemplary embodiment, the aforementioned temporal periods are measured from the point in time where an amount of power has been transferred is that which results in at least more than and/or equal to an elevation of a state of charge by at least and/or equal to 60, 65, 70, 75, 80, 85, or 90% or more of a state of charge of an implanted power storage device, such as a battery, and/or raising the state of charge thereto, during an action of transferring power from the apparatus to the implanted medical device, has occurred.

In view of the above, it can be seen that in an exemplary embodiment, the action of cooling of method action 1230 is not executed during the action of transferring power. In an exemplary embodiment, no active cooling is executed during the action of transferring power (as distinguished from cooling which might occur due to ambient conditions, such as, for example, when an ambient room temperature/air temperature is 20° C. when a surface of the external component, such as the surface that faces away from the skin of the recipient/the surface on the opposite side of the skin interface surface, is at 30° C., for example).

In an exemplary embodiment, there is the additional method action of, after at least a temporal majority of the action of active cooling (e.g., 50.1% of the total time), or after at least 55, 60, 65, 70, 75, 80, 85, 90, 95, or 100%, or any value or range of values therebetween in 1% increments of the temporal period of the action of active cooling has elapsed, recharging the transcutaneous power transfer apparatus to enable such to execute the action of transferring power from the apparatus to the implanted medical device, wherein a temperature of a skin interfacing surface of the transcutaneous power transfer apparatus is at or below a safety value temperature sufficient to enable safe interface with skin of the recipient after the action of recharging is complete, due to the action of cooling.

In an exemplary embodiment, the action of recharging the implant battery is executed such that a state of charge of the battery or otherwise a power storage device of the external component/transcutaneous power transfer apparatus is increased by at least 30, 35, 40, 45, 50, 55, 60, 65, 70, 75, 80, 85, 90, 95, or 100%, or any value or range values therebetween in 1% increments or to a state of charge corresponding to such. In an exemplary embodiment, all active cooling is executed prior to reaching any one or more of those values.

In an exemplary embodiment, with reference to the scenario where 100% of the temporal period of active cooling has elapsed (i.e., active cooling is completed), recharging then commences. Charging does not occur during the period of active cooling in this embodiment. That said, in an alternative exemplary embodiment, such as with respect to the scenario where 70% of the temporal period of active cooling has elapsed, recharging could then commence, and thus there would be a period of recharging that overlaps with the period of active cooling. That said, in an exemplary embodiment, cooling could be suspended during recharging, and then cooling could be recommenced to take up the remaining 30% for example. Moreover, in an exemplary embodiment, such as where the recharging is rapid, the recharging could take place during two-thirds of the remaining 30%, and then 10% of the period of cooling would be located after recharging ends.

In an exemplary embodiment, after at least a temporal majority of the action of cooling (or after any of the aforementioned percentages of the temporal period of cooling) or before at least a temporal majority of the action of cooling (or before at least 5, 10, 15, 20, 25, 30, 35, 40, 45, or 50% of the period, or any value or range of values therebetween in 1% increments), the method includes commencing recharging or completing recharging of the transcutaneous power transfer apparatus to enable such to execute the action of transferring power from the apparatus to the implanted medical device, wherein a temperature of a skin interfacing surface of the transcutaneous power transfer apparatus is at or below a safety value temperature sufficient to enable safe interface with skin of the recipient immediately after the action of recharging is complete in the scenario where recharging is executed after at least the temporal majority or immediately after the action of cooling is complete in the scenario where recharging is executed before the temporal majority, due to the action of cooling.

In an exemplary embodiment, the action of transferring power in method action 1220 is executed as part of a fast charge of the implant, wherein heat generated by the apparatus as a result of the fast charge is absorbed by a heat absorbing arrangement of the apparatus (e.g., the thermal mass detailed below), thereby preventing a skin interface surface of the apparatus from exceeding a temperature beyond that which would otherwise be the case, which temperature would be at least uncomfortable for the recipient. Additional details of this are described below.

Fast charge is distinguished from normal charging. Because of the rapid discharge of the battery that would result and/or because of the higher load on the coil driver, the temperature of the external component would increase relative to that which would be the case if a less rapid charging regime was utilized.

An exemplary embodiment of an exemplary method further includes the action of charging the implanted prostheses during a non-fast charge action. In such a scenario, the action actively cooling is not executed in conjunction with the non-fast charge action. In this regard, in an exemplary embodiment, the teachings herein can be applied in a controlled or otherwise limited manner when “necessary” (very loosely termed with respect to some embodiments or otherwise scenarios) and otherwise not utilized when not necessary. Accordingly, in at least some exemplary embodiments, there are method actions disclosed herein that are executed in conjunction with not actively cooling the external component. In an exemplary embodiment, any one or more of the method actions detailed herein not associated with cooling (e.g., placing external component on the skin/executing recharging of the implant, etc.) are executed at least after and/or before 3, 4, 5, 6, 7, 8, 9, 10, 11, 12, 13, 14, 15, 16, 17, 18, 19, or 20 hours, or 1 or 2 or 3 or 4 or 5 days of affirmative cooling of the external component.

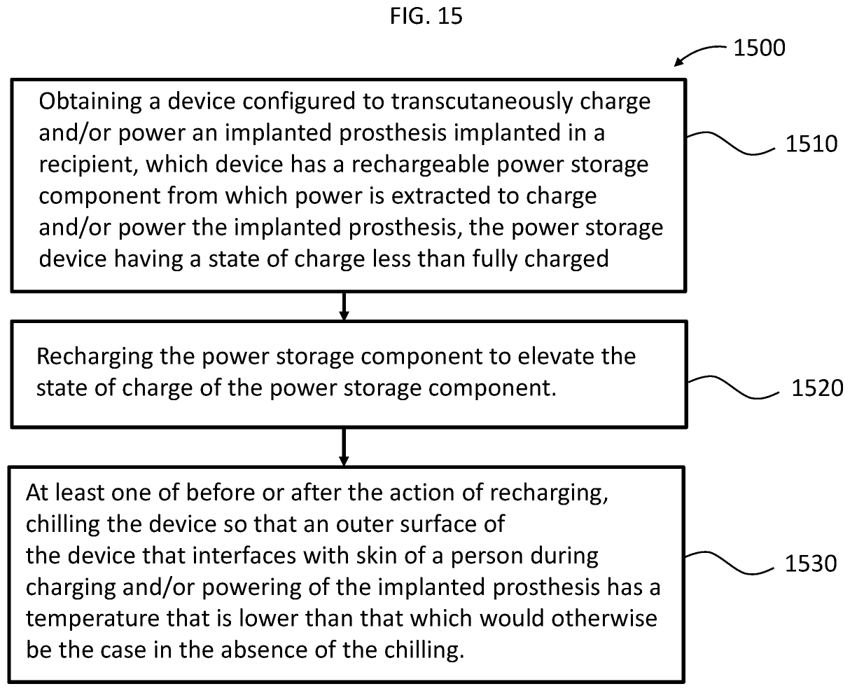

FIG. 15 presents an exemplary algorithm for another exemplary method, method 1500, which includes method action 1510, which includes obtaining a device configured to transcutaneously charge/recharge and/or power an implanted prosthesis implanted in a recipient, which device has a rechargeable power storage component (e.g., battery) from which power is extracted to charge/recharge and/or power the implanted prosthesis, the power storage device having a state of charge less than fully charged/a state of charge where there is utilitarian value in recharging This can be the off the ear chargers detailed above, or can be other devices, such as an off the ear sound processor, etc.

Method 1500 further includes method action 1520, which includes the action of recharging the power storage component to elevate the state of charge of the power storage component. This can be done with the device of FIG. 14 (again, more on this below) by way of example only and not by way of limitation. This can be also done with other devices, such as a dedicated charger that does not have the cooling features associated with the embodiment of FIG. 14. This can be a charger that relies upon a wired connection with the device configured to transcutaneously charge and/or power and/or can be a charger that utilizes a wireless link, such as the inductance field detailed above, to charge the device.

Method 1500 further includes method action 1530, which includes the action of at least one of before or after the action of recharging, chilling the device (as distinguished from the scenario where the device simply cools because the ambient temperature is lower than the device) so that an outer surface of the device that interfaces with skin of a person (e.g., skin interface surface 690) during charging and/or powering of the implanted prosthesis has a temperature that is lower than that which would otherwise be the case in the absence of the chilling. In an exemplary embodiment, the action of chilling the device is executed prior to the action of recharging, while in another exemplary embodiment, the action of chilling the device is executed after the action of recharging. Doing one does not exclude doing the other. Still, in some embodiments, only one is done and not the other. As detailed above, in some embodiments, the action of chilling is executed using a dedicated cooling apparatus (perhaps more appropriated called a chilling apparatus) separate from the device. In some embodiments, the dedicated cooling apparatus is also a recharging apparatus configured to recharge the device to enable such to execute the action of transcutaneous charging and/or powering the implanted prosthesis.

While the preceding paragraph focuses on reducing the temperature or otherwise managing the temperature of the skin interface surface, it is noted that in other embodiments, the focus can be on other components of the external device. By way of example only and not by way of limitation, this can be the thermal mass as detailed below.

In an exemplary embodiment, the action of chilling the device is executed using passive heat exchange from the device. This as distinguished from using the arrangement of FIG. 9, for example.

As noted, in an exemplary embodiment, the action of chilling the device (the transcutaneous power transfer apparatus), such that it is below the ambient temperature (or a specific component, such as the skin interface surface or another component thereof), is executed using a dedicated cooling apparatus separate from the transcutaneous power transfer apparatus. FIG. 14 depicts an exemplary embodiment of this separate cooling apparatus. FIG. 14 provides an exemplary device 2000 that can enable some of the teachings detailed herein during the action of charging an external component that is utilized to charge or otherwise provide power to an implantable component, such as by way of example only and not by way of limitation, the external component 640 (the transcutaneous power transfer apparatus of method action 1210), which is shown in FIG. 12 as being located in the charging device 2000 in a position for charging. The charging device 2000 is configured to inductively charge the external component utilizing an inductance coil charging device 2042, which charging device can correspond to or otherwise have the components that are similar to or otherwise analogous to the components that are utilized by the external component to charge the implant. That said, in an alternate embodiment, a hardwired system including a plug that plugs into the external component 640 can be utilized to charge the external component 640. That is, inductance charging is but one option, and a more traditional method of charging utilizing the direct flow of electrons the battery of the external component can be utilized in some other embodiments. Moreover, it is noted that the coils used for inductance communication with the implant may not necessarily be used for recharging. In some embodiments, a second coil for recharging is present and/or additional circuitry is used to convert the AC current to the DC current (so that a different recharging frequency (from the power transmission to the implant frequency) can be used, for example). The additional coil can be co-located with the transfer coil, or could be located away from the coil (e.g., on the opposite side of the headpiece—in which case external component 640 would be shown in FIG. 2 upside down from that shown).

In an exemplary embodiment, a method of using charging device 2000 includes placing the transcutaneous power transfer device into the receptacle of the charging device 2000. Then, the transcutaneous power transfer device is charged in the receptacle, and a charging device controlled charging process can be executed. That is, there are electronics inside/as part of/co-located with the charging device which control start, execution and end of the charging process of the transcutaneous power transfer device. This can also control the rate of charge, and other charging parameters. This can be a circuit based control device, a microprocessor controlled device, or a timer based control device. The charging device can include programming, stored in a memory, that can initiate and/or terminate and/or regulate and otherwise control the recharging process (and cooling/chilling process, as will be detailed below). This programming can be accessed or otherwise can be used to control the microprocessor or other processors of the device 2000. Still, in other embodiments, solid-state or semi solid state electronic circuits can be utilized to execute the functions that would otherwise be executed by the programming. Standard timers can be utilized to initiate and terminate the charging, etc. Sensors can be utilized to determine the state of charge of the battery the external component so as to control the charging/recharging process.

The charging/recharging includes wired or wireless communication between the transcutaneous power transfer device and the charging device. Charging of the transcutaneous power transfer device is initiated by one of the following: (1) automatically, once the transcutaneous power transfer device is placed into the receptacle, through corresponding detection sensors built into the receptacle, etc., (2) by pressing a button or providing some other input via an input suite of the charger to initiate the charging process/control the charging process. The cooling/chilling feature of the charging device is used to remove excess heat generated during recharge of the charger battery. This can be done after recharging, whether after all charging is completed, or after recharging has commenced (so there is overlap). That said, in an exemplary embodiment, the cooling/chilling is utilized to chill the device or otherwise cool the device below ambient temperature so that the heat generated during the charging/recharging process results in an external component that has a cooler temperature than that which would otherwise be the case at the end of recharging of the external component, and thus the external component has a cooler temperature than that which would otherwise be the case at commencement of recharging of the implant.

In an exemplary embodiment, the transcutaneous power transfer device is thus pre-cooled in the receptacle of the charging device to a temperature that is below the average core body temperature, prior to using the transcutaneous power transfer device to charge/recharge the implant. Pre-cooling can be initiated by at least one of the following: (1) automatically, once recharging of the power transfer device is complete; or (2) by pressing a button on the recharger or using some other input mechanism to initiate the pre-cooling process. When the transcutaneous power transfer device has reached a target temperature (whether by active temperature sensors (thermocouples or IR, etc., for example) or the use of latent variables (e.g., time could be used, alone or coupled with other data, such as known ambient air temperature (which could be obtained from a temperature sensor or from the internet for example or from a household temperature system, autonomously or by manual input), the charging device can indicate via visible or audible notification that the transcutaneous power transfer device is ready to use.

In a third action, the pre-cooled transcutaneous power transfer device is used to rapidly complete a recharge of the implanted battery. Heat generated during the recharge is at least in part absorbed by the pre-cooled transcutaneous power transfer device in general, and the dedicated thermal mass in some embodiments, and thus is not transferred to the skin of the recipient/less of such generated heat is transferred to the skin. In a fourth action, the now warmed-up transcutaneous power transfer device is returned to the battery charger and the process is repeated.

In an exemplary embodiment, all things being equal, an amount of thermal energy transferred to skin of the recipient for any one or more of the charging regimes of the implant detailed herein is at least or equal to 20, 25, 30, 35, 40, 45, 50, 55, 60, 65, 70, 75, 80, 85 or 90% less or any value or range of values therebetween in 1% increments than the net transfer that would otherwise be the case in the absence of the cooling/chilling teachings detailed herein. These aforementioned values are related to the entire recharging. In fact, in some embodiments, heat transfer actually occurs from the skin to the device. In any event, in an exemplary embodiment, during the first period of recharging, heat transfer could be to the device, and then in the second period of time, after the device warms up, heat transfer could occur to the human. Still, in some embodiments, the total amount of the heat transfer into the device could be at least or equal to 5, 10, 15, 20, 25, 30, 35, 40, 45, 50 or more or any value or range of values therebetween in 1% increments of the amount of heat transfer that would have otherwise existed out of the device, again all things being equal.

The transcutaneous power transfer device has various constructive and physical properties that are aimed at maximizing or otherwise increasing the transfer and conversion of heat energy emitted from components of the transmission pathway used to fast-charge the implant. At the same time, an overall safe temperature regime is maintained at all times during the fast-charging process until the depleted implanted battery is fully charged (or otherwise charged to the maximum desirable (e.g., 80% to prolong battery life), and the fast-charging process has thus been completed.

It is briefly noted that herein, cooling will be referred to sometimes in place of chilling, and vice versa. Any disclosure of one corresponds to a disclosure of the other. Herein, unless otherwise specified, cooling and chilling are used to cover cooling and chilling beyond mere stabilization of temperatures due to an ambient temperate (beyond mere assuming room temperature).

Some additional details of the charging apparatus of FIG. 14 will be described below. Now, additional features of method 1500 will be described.

In some embodiments, the action of recharging is executed as part of a fast recharge of the device, wherein heat generated by the device as a result of the fast charge is absorbed by a heat absorbing arrangement of the device (again, more on this below), thereby preventing a skin interface surface of the device from exceeding a temperature beyond that which would otherwise be the case, which temperature would be at least uncomfortable for the recipient. In some instances, the action of chilling is not executed during the action of recharging, while in other instances, the action of chilling overlaps with the action of recharging. In the interests of textual economy, all of the above noted features regarding chilling and recharging associated with the implanted battery of the implant are, in some embodiments, applicable to the battery/power storage device of the device configured to transcutaneous charge and/or power the implanted prosthesis, and vice versa. In an exemplary embodiment, after at least a temporal majority of the action of chilling or before at least a temporal majority of the action of chilling, commencing the action of recharging the device to enable such to execute an action of transferring power from the device to the implanted prosthesis, wherein a temperature of a skin interfacing surface of the device is at or below a safety value temperature sufficient to enable safe interface with skin of the recipient after the action of recharging is complete in the scenario where recharging is executed after at least the temporal majority, or after the action of chilling is complete in the scenario where recharging is executed before the temporal majority, due to the action of chilling. Again, consistent with embodiments where the teachings above associated with the implant recharging can correspond to the charger recharging, the action of chilling can overlap with the action of recharging, while in other embodiments, it does not overlap.

In an exemplary embodiment, in view of FIG. 14, the action of chilling the transcutaneous power transfer apparatus/external device below the ambient temperature is executed using a dedicated cooling apparatus separate from the transcutaneous power transfer apparatus/device, and the dedicated cooling apparatus is also a recharging apparatus configured to recharge the transcutaneous power transfer apparatus to enable such to execute the action of transferring power from the apparatus to the implanted medical device. This is in contrast to, for example, the embodiments where the action of chilling the transcutaneous power transfer apparatus/external component is executed utilizing a dedicated cooling apparatus separate from the transcutaneous power transfer apparatus that is not also a recharging apparatus, where such dedicated cooling apparatus could be, for example, a refrigerator, such as a standard household refrigerator. In an exemplary embodiment, the refrigeration section can be used, and in an alternate embodiment, the freezer section can be used. In an exemplary embodiment, the external device/transcutaneous power transfer apparatus can be placed into a refrigerator section or the freezer section for less than or equal to or greater than 5, 10, 15, 20, 25, 30, 35, 40, 45, 50, 55, 60, 65, 70, 75, 80, 85, 90, 95, 100, 110, or 120 minutes, or any value or range of values therebetween in 1 minute increments. In an exemplary embodiment, a holding device/support device can be utilized to hold and/or support the device while the external device is in the refrigerator/freezer. In an alternate exemplary embodiment, the external component is placed into the refrigerator/freezer without a support, although perhaps a paper towel or the like can be placed underneath the external component. In this regard, in an exemplary embodiment, the method can entail placing the external component/transcutaneous power transfer apparatus into the refrigerator or freezer to chill the device and otherwise execute method action 1530, and then transfer the power from the apparatus to the implanted medical device with the external component at the artificially reduced low-temperature relative to that which would be the case with respect to ambient. (This can also be done after the power transfer action is executed.)

In an exemplary embodiment, the recharging can take place while the external component is in the refrigerator or freezer. In this regard, in an exemplary embodiment, a battery-powered recharger could be connected to the external component and the battery-powered recharger and the external component can both be placed in the refrigerator and/or freezer, at least embodiments where simultaneous cooling/chilling are to be utilized.

In an exemplary embodiment, the action of chilling the external device/the transcutaneous power transfer apparatus can be executed by placing a cold pack or a freeze pack against a surface of the external component (or multiple packs can be used, paced on different respective surfaces) so as to actively cool that component prior to transferring power (or after transferring power). By way of example only and not by way of limitation, in an exemplary embodiment, there can be a heat transfer path/a route for thermal transfer from the external surface to the thermal mass briefly noted above (and additional details of which will be provided below—this section is about method actions) that will speed up the process of chilling the thermal mass, and thus the transcutaneous power transfer apparatus/external device. This can also be the case with respect to the embodiment associated with utilizing the freezer or the refrigerator. The surface that is in thermal communication with the thermal mass can be utilized for convective heat transfer and/or radiative heat transfer to the ambient environment inside the refrigerator or freezer. In an exemplary embodiment, the aforementioned holding device and/or support device can have fins or the like that will increase the rate of heat transfer from the external device in general, and the thermal mass in some embodiments that utilize such. That is, for example, a thermal conductive path can extend from the thermal mass to the external surface, where the external surface is in contact with a surface of the support device which is in thermal communication with fins or other heat transfer enhancing devices of the support device.

In an exemplary embodiment of the method 1500, the action of chilling the transcutaneous power transfer apparatus is executed by transferring heat away from a thermal mass of the apparatus. In an exemplary embodiment of this exemplary embodiment, the thermal mass is present for the purpose of maintaining a temperature of a skin interface surface of the apparatus at a level below that which would otherwise be the case in the absence of the thermal mass. By way of example only and not by way of limitation, an aluminum solid body or a steel or a titanium solid body can be utilized as the thermal mass. It is noted that the high thermal mass material can be made out of aluminum, steel, or titanium, or some other material that has utilitarian value with respect to the thermal mass embodiments.

Additional details of this embodiment will be described below, but briefly, the idea is that by utilizing a body that has a high thermal captivation feature, beyond that which is the case for the general components of the transcutaneous power transfer apparatus (this is an additional element specifically designed/purposed for this method), this body can absorb the heat that is generated during the action of transferring power from the apparatus to the implanted medical device, and thus achieve a phenomenon where the surface temperature of a skin interface surface of the external component/transcutaneous power transfer apparatus is kept lower relative to that which would otherwise be the case in the absence of this thermal mass. Again, more on this below.

In an exemplary embodiment, the transcutaneous power transfer device includes a charge regulation/output control device, which can be microprocessor based and/or can be circuit based, which circuits are specifically designed to regulate charging/recharging. In this exemplary embodiment, the controller receive input indicative of a temperature of a component of the power transfer device, such as, for example, the skin interface surface, or other component that can have utilitarian with respect to gauging the skin interface surface temperature, and use that input to control or otherwise regulate the charging. If a temperature above a certain level and/or if a rate of change of temperature meets a certain threshold, the charging regime can be adjusted, at least temporarily, to prevent certain temperatures from being reached or otherwise reduce the rate of temperature increase.

In an exemplary embodiment, when the transcutaneous power transfer device is in a fast charging arrangement, fast charging can be limited per a state of charge measurement, such as, for example, coulomb counting or any other arrangement that can enable such. In this regard, there can be SOC-determining electronics inside/with the implant, and this information can be communicated to the external device. That said, in an exemplary embodiment, latent variables can be utilized or otherwise an estimate of the state of charge can be made of the battery of the implanted device.

In some embodiments, there is a device comprising an inductive power transmission system configured to transfer power to an implanted medical device, a skin interface surface, and a dedicated passive conduction thermal transfer apparatus configured for temperature management of the device during the transfer of power. This as contrasted to the active cooling systems of FIG. 8 for example (and as also contrasted to natural heat transfer that occurs with all objects, which is neither dedicated nor management). In an exemplary embodiment, with reference to the thermal mass noted above by way of example only, the dedicated passive conduction thermal transfer apparatus is a dedicated thermal mass configured for thermal mass cooling of the device.

It is noted that the passive conduction thermal transfer apparatus can also utilize convention features and radiative features. It is just that the device operates on conduction principles in a utilitarian manner that is not trivial or incidental.

It is briefly noted that in some embodiments, the heat is generated by the battery during charging and/or discharging. It is further noted that in some exemplary embodiments, the heat is generated by the inductance coil. Embodiments include thermal masses that absorb heat generated by one or both of these devices.

Embodiments that utilize the thermal masses operate on the principle of not transferring thermal energy out of the device, but transferring it to another location within the device. Or more accurately, in some embodiments, transferring more thermal energy to another location within/that is part of the device beyond that which would otherwise be the case. In this regard, by way of example only and not by way of limitation embodiments include transferring at least or more than 25, 30, 35, 40, 45, 50, 55, 60, 65, 70, 75, 80, 85, or 90% or more, or any value or range of values therebetween in 1% increments of the overall heat generated during a charging/recharging process of an implant to a dedicated thermal mass, wherein the charging/recharging process and correspond to any of those detailed herein.

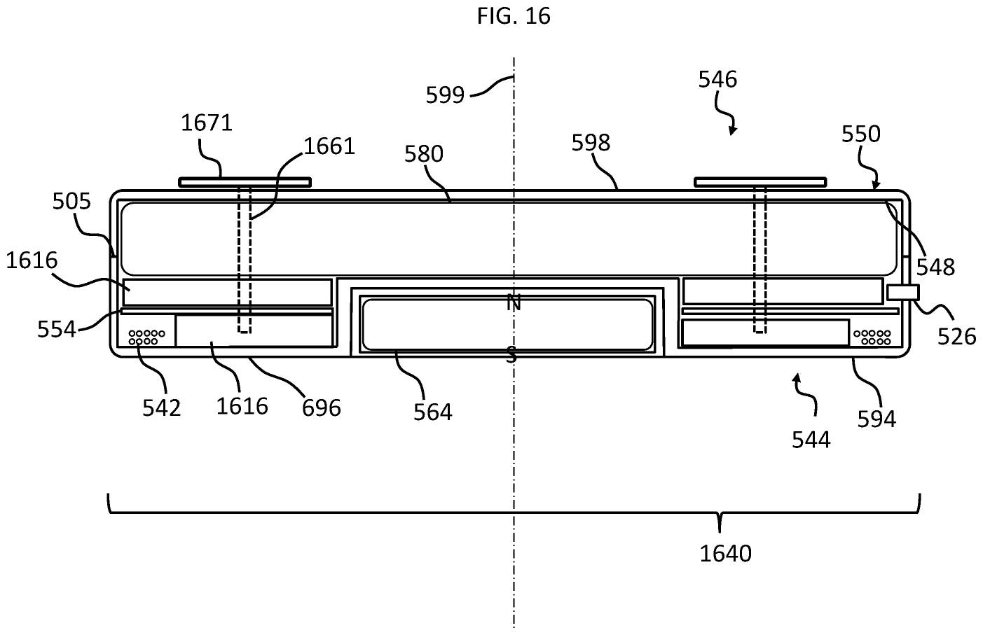

In this regard, FIG. 16 presents an exemplary external component 1640. As can be seen, thermal mass(es) 1616 are located on the bottom wall of the housing, and above such, which bottom wall establishes the skin interface surface 696. These thermal masses can be aluminum or some other body. In this exemplary embodiment, the masses are connected to heat sink 1671 via thermal conductor 1661 (which could be a copper cylinder or a fluid filled tube that is thermally conductive), which corresponds to a radiator device (in an embodiment, the ice pack is placed onto these sinks 1671). In this exemplary embodiment, conductive paths 1661 can extend through the battery or can extend around the battery to reach the opposite side 546. The housing facing side of heat sinks 1671 can be above the top surface of the housing to enable airflow between these two components to increase the transfer therefrom.

It is noted that in an alternate embodiment, the heat sinks 1671 can additionally or instead extend about the outer periphery of the external component 1640 (this could be a band that extends concentrically about the axis 599), which component is in heat transfer communication with the masses 1616.

It is noted that with respect to at least some embodiments utilizing the thermal mass, there can be utilitarian value with respect to protecting the thermal mass from radiative and/or conductive and/or convective heat transfer with the ambient environment after the thermal mass has been chilled or otherwise actively cooled, such as chilled or cooled below ambient temperature. In this regard, embodiments include utilizing the thermal mass as a heatsink to absorb the thermal energy generated during the charging, such as the fast charging, so that the skin interfacing surface 696 does not heat up to a temperature that is at or above a non-utilitarian value. In this regard, the heat sinks 1671 could provide a path for heat from the ambient environment to travel to the thermal masses 1616. Thus, the “efficiency” of the heat management techniques applied to the external device would be less than that which would otherwise be the case, because the ambient temperature would include a “heating” component to the thermal mass, thus resulting in less “room” if you will for absorption of thermal energy generated during the charging/recharging processes. Put another way, in at least some exemplary embodiments, the sole purpose of the thermal masses is to absorb thermal energy generated during the charging/recharging (charging the battery 580 or using the battery to charge an implant) process, and the cooler that the thermal mass remains at the commencement of charging and during that process, the more efficient the management of the skin interfacing surface temperature will be.

Thus, embodiments include arrangements that thermally insulate the thermal mass from the ambient environment, as distinguished from the heat generating environment of the external component in general, and the heat generating portions of the external component associated with charging and/or recharging, in particular. Thus, in some exemplary embodiments, the device is a thermally insulated device (meaning it is insulated beyond that which would be the case with respect to a general housing arrangement that would be used in any standard device), which can be counterintuitive with respect to concepts of increasing heat transfer from the external device. That is, instead of arrangements that are designed to establish ease of thermal transfer between the device and the ambient environment, embodiments include arrangements that reduce the amount of thermal transfer that exists between the device and the ambient environment relative to that which would otherwise the case in the absence of these arrangements.

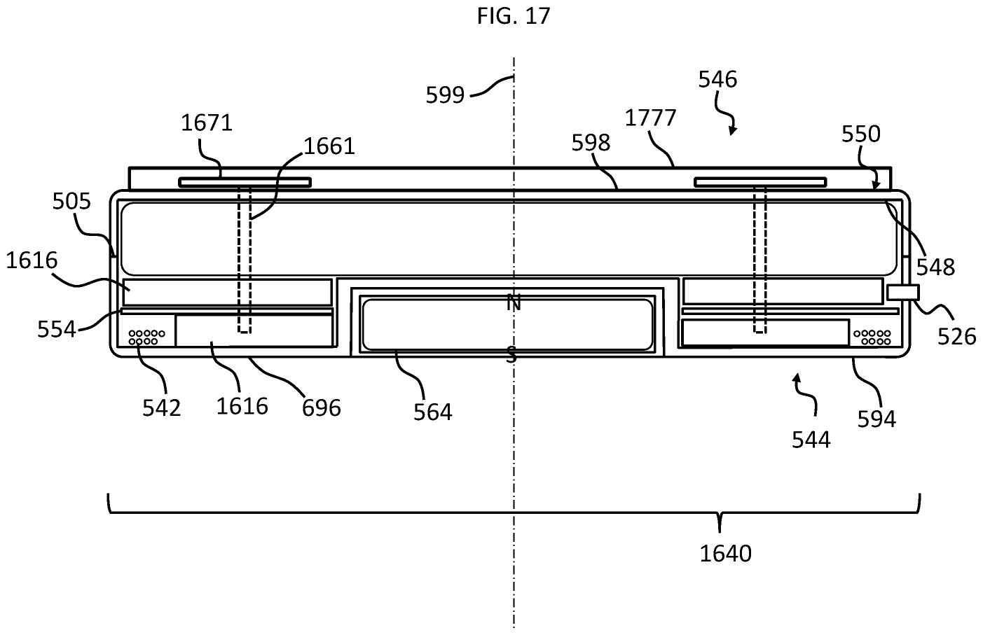

FIG. 17 provides a simple exemplary embodiment where a thermally insulative cap 1777 is placed over the heat sinks 1671 when the external component 1640 is being utilized to charge an implant, for example. Here, the utilitarian value with respect to having the heat sinks 1671 so as to increase the rate of chilling or cooling of the thermal masses according to the exemplary methods detailed above can be retained, and then, after chilling or cooling has been achieved, the insulative cap 1777 is placed over the heat sinks 1671, thus thermally insulating those heatsinks from the ambient environment, and thus reducing the amount of heat transfer from the ambient environment into the now chilled thermal masses relative to that which would otherwise be the case.

In an exemplary embodiment, the sidewalls, or at least a portion thereof, of external device of 640 FIG. 6 and/or the top wall and/or the bottom wall are made of metal, such as aluminum or steel or titanium, or some other high thermal mass material, instead of a polymer such as plastic or the like. An exemplary embodiment, the housing, or at least a portion thereof, of the embodiment of FIG. 6 or FIG. 5 for that matter can be made of metal instead of plastic. Because some embodiments may not desire to have a metal contacting the skin of the recipient, a polymer or otherwise nonmetallic skin interface body can be located at the bottom of the housing between the metal and the skin. That said, in an exemplary embodiment, the bottom of the housing is made of a polymer or otherwise not made of a metal. This can also extend up the sides, at least for a millimeter or 1.5 or 2 or 2.5 mm or more or so, so that any skin deformation that results in wraparound will still not result in skin to metal contact.

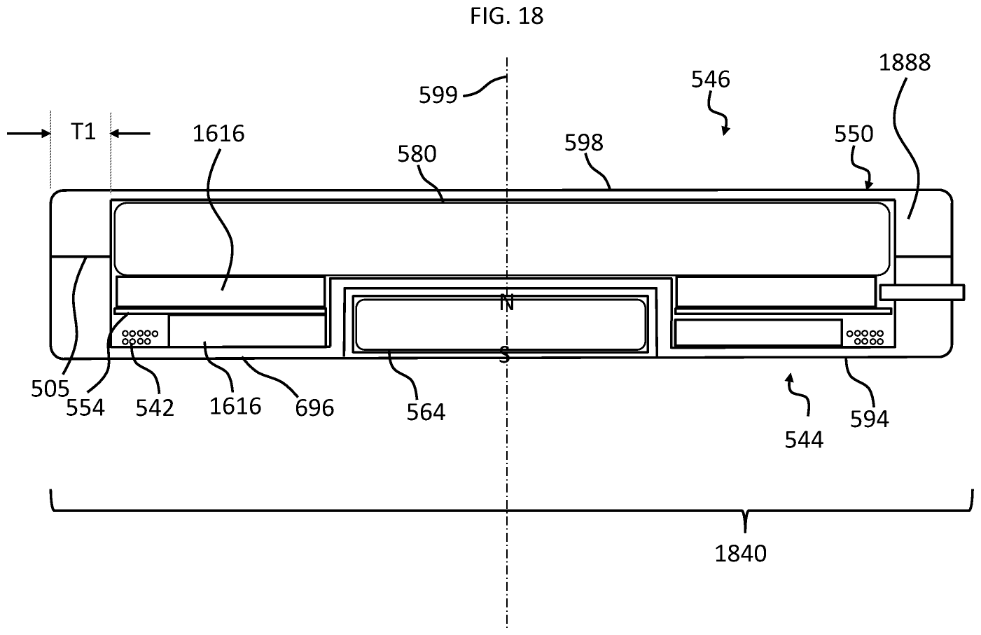

Some more specifics of this concept will be described in terms of FIG. 18. FIG. 18 presents an exemplary embodiment of an external device 1840 configured for transcutaneous transfer of power to an implantable device which includes the aforementioned thermal masses 1616 of FIG. 16 and the additional thermal mass of wall 1888. Here, the thickness of wall 1888 is shown to be larger than that which is the case with respect to the embodiments of FIGS. 5 and 6, etc., above. However, it is noted that the teachings of FIG. 18 can correspond to a wall thickness corresponding to those of FIGS. 5 and 6, except that here, the figure simply shows a thicker wall for purposes of visualization of the thickness measurement lead lines. In any event, here, the wall is a thermal mass in addition to a wall. The material of the wall can be a metal that has utilitarian thermal mass properties relative to that which would otherwise be the case with respect to a wall of the embodiment of FIG. 5 when such is a polymer. In some embodiments this is achieved via the overall thickness, which thus increases the mass, and in some embodiments this is achieved by the material selection (indeed, in some embodiments the wall thickness may be thinner than that of the polymer of the embodiment of FIG. 5 when such is used), and in some embodiments it is both. By way of example only and not by way of limitation, in an exemplary embodiment, instead of a polymer wall, the wall is made out of aluminum or some other material having utilitarian value with respect to achieving the utilitarian value of the thermal mass. In an exemplary embodiment, the wall can be painted and/or plated with another material having a more aesthetically pleasing feature.

It is briefly noted that in the embodiment of FIG. 18, the top thermal masses 1616 are in direct contact with the battery 580. This creates a thermal conductive path (face to face direct contact) between the thermal mass and the battery 580. This enhances heat transfer from the battery to the thermal mass 1616. It is also noted that in an exemplary embodiment, a thermal conductivity path can be located between the top thermal mass 1616 and the bottom thermal mass 1616. This can be a cylinder of aluminum or copper or some other conductive material. In an exemplary embodiment, the thermal mass 1616 at the top is monolithic with that of the bottom, or otherwise in direct contact, and such direct contact is achieved via a passage through the board 554. In an exemplary embodiment, there can be a space and/or insulation between the battery and the thermal mass at some locations.

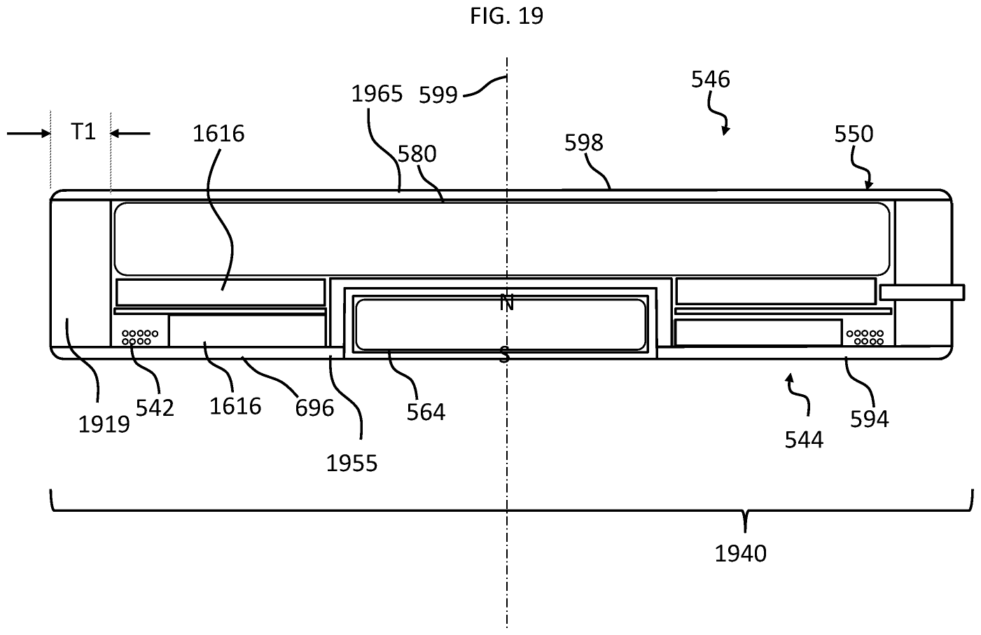

The embodiment of FIG. 18 depicts a housing that is made out of the material of the wall 1888. In this regard, in an exemplary embodiment, the housing is made out of aluminum instead of a polymer. FIG. 19 presents another exemplary embodiment of an external device, 1940, where the housing is a composite housing, where the top and bottom portions (the bottom portion establishing the skin interface surface) is made of a polymer, and the sidewalls are made of the aluminum. This can have utilitarian value with respect to avoiding direct contact with the thermal mass and the skin of the recipient. As seen, the top and bottom portions of the housing, portions 1965 and 1955 respectively, are made out of a polymer that is conducive or otherwise skin friendly or otherwise comfortable or more comfortable with respect to skin contact, and the sidewalls 1919 are made out of the thermal mass material, such as aluminum. Utilizing the bottom wall constructed out of a polymer, the material of the sidewalls 1919 are maintained away from direct contact with the skin. With respect to the embodiment of FIG. 19, in some embodiments, the bottom wall 1955 is made of the polymer or otherwise skin friendly or comfortable material, and the remainder of the housing is made of the thermal mass material. That is, in an exemplary embodiment, 1965 can also be made of the same material as the wall 1919. The top may be monolithic with the sidewalls in some embodiments.

Owing to the relative greater thickness of the sidewalls T1 relative to the exemplary sidewalls detailed above with respect to the embodiment of FIG. 5 for example, the thermal mass features can be enhanced. That said, in some embodiments, the thickness of the sidewalls can be the same as the embodiment of FIG. 5 for example, such as in embodiments where it is the material that is utilized in lieu of the amount of material.

In an exemplary embodiment, T1, as measured in a direction normal to the longitudinal axis 599, is greater than or equal to 0.5, 0.75, 1, 1.25, 1.5, 1.75, 2, 2.5, 3, 3.5, 4, 4.5, 5, 5.5, 6, 6.5, 7, 7.5 8, 8.5, 9, 9.5, or 10 mm or more, or any value or range of values therebetween in 0.1 mm increments. In an exemplary embodiment, the height of the wall (1919 or 1888) is greater than or equal to 4, 5, 6, 7, 8, 9, 10, 11, 12, 13, 14, 15, 16, 17, 18, 19, or 20 mm or more, or any value or range of values therebetween in 0.1 mm increments. It is also noted that the thickness of the sidewall need not necessarily be symmetric. The sidewall can be thicker on one side versus the other.

It is also noted that the aforementioned thicknesses, at least some of them, can correspond to the thickness of the top and/or bottom wall as well.

In an exemplary embodiment, the thermal mass is at least and/or equal to 150, 200, 300, 400, 500, 600, 700, 800, 900, or 1000 percent or more, or any value or range of values therebetween in 1% increments above that of a polymer plastic, or above PEEK or above ABS or above polycarbonate.

In an exemplary embodiment, the device 1940 (and the other external devices for that matter) has a circular or substantially circular shape or an oval shape or an egg shape when viewed from the top (looking down with respect to the view of FIG. 19/looking down along the longitudinal axis 599 at surface 598) having outer diameter of greater than or equal to 35, 36, 37, 38, 39, 40, 41, 42, 43, 44, 45, 46, 47, 48, 49, 50, 51, 52, 53, 54, 55, 56, 57, 58, 59, or 60 mm or more, or any value or range of values therebetween in 0.1 mm increments. In an exemplary embodiment, for example, an outer diameter can be 40 mm and a diameter as measured normal to that measurement can be 50 mm. It is also noted that all of the aforementioned outer diameter values (or thickness values, in fact) can correspond to any cross-section taken on any plane normal to the longitudinal axis 599.

It is noted that while the embodiment of FIG. 19 or the embodiment of FIG. 18 include the thermal masses 1616, in some embodiments, these may not necessarily be present. That is, instead, thermal mass of the wall 1919 or 1888 is utilized.

As seen, embodiments utilize the magnet 564. In an exemplary embodiment, the magnet can also be included in the overall thermal masses. This can be especially the case if the magnet is placed into increased thermal conductivity with respect to the generating components/components that generate heat during charging and/or recharging, as opposed to a thermally isolated magnet. In this regard, in at least some exemplary embodiments, the magnet 564 is not thermally insulated from the heat generating components. Thus, in some embodiments, a thermally conductive path is present between the heat generating components and the magnet(s). That said, in some embodiments, the magnets are purposely thermally insulated from the heat generating components. This can have utilitarian value with respect to embodiments where the magnet is not to be used as a heatsink or otherwise a thermal mass with respect to absorbing thermal energy from the heat generating components. In an exemplary embodiment, the magnet could potentially be maintained or otherwise protected from heating and otherwise retain at a lower temperature/the chilled or cooled temperature, so as to obtain an overall lower temperature on the skin interface surface relative to that which would otherwise be the case if the magnet was in thermal conductivity with the heat generating components/was not thermally insulated therefrom.

And the features relating to thermal conductivity with the heat generating components and the thermal mass are also applicable to, for example, the thermal masses 1616 and 1919 and 1888. Again, it is noted that in some embodiments that utilize the wall thickness or otherwise establish the wall as a thermal mass, the separate thermal masses 1616 may or may not be present. It all depends on the amount of thermal mass and/or the properties thereof in the overall utilitarian value with respect to the increased mass and the thermal management techniques detailed herein.

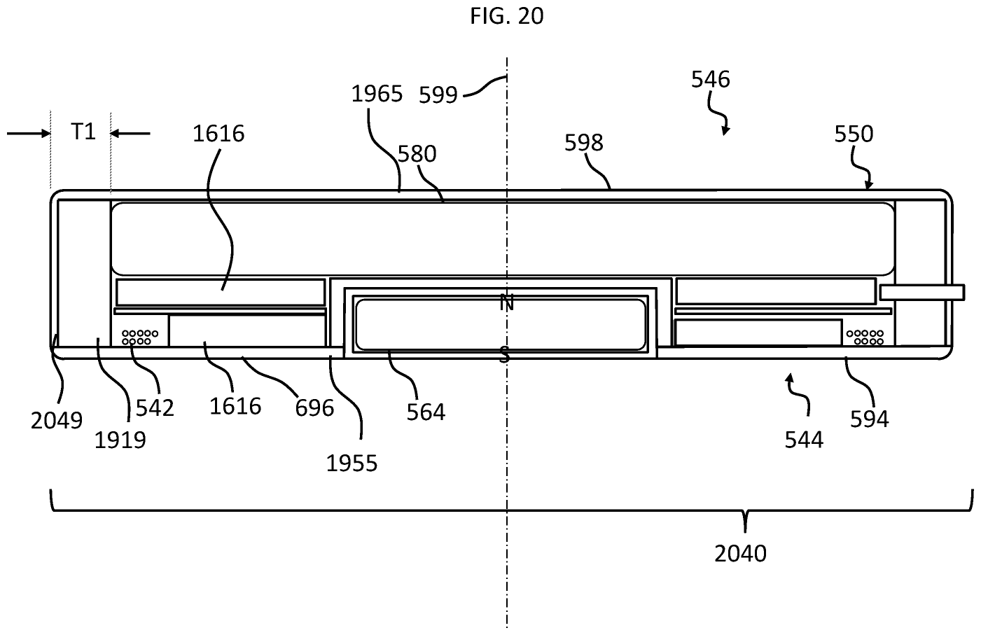

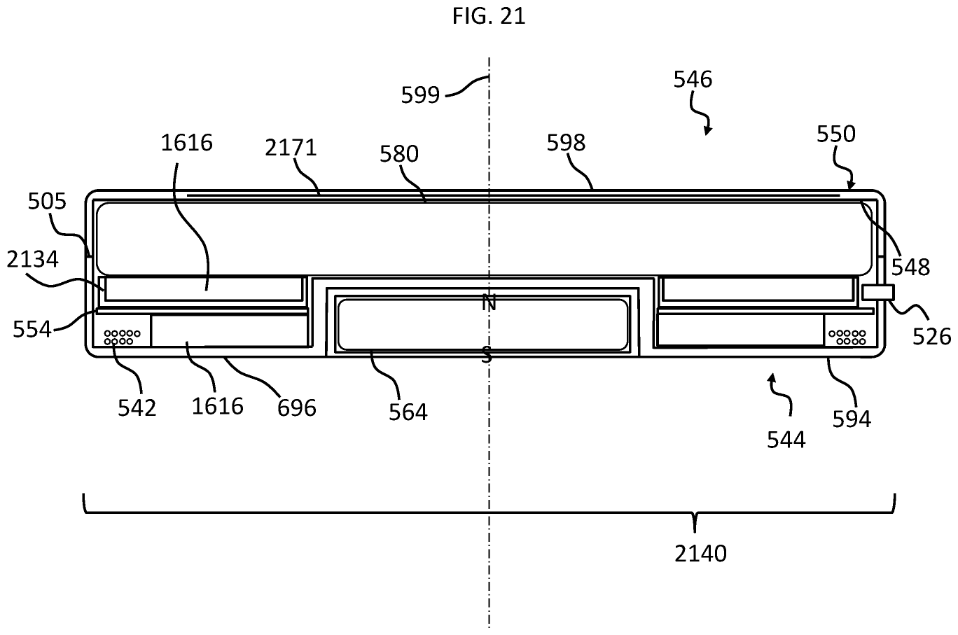

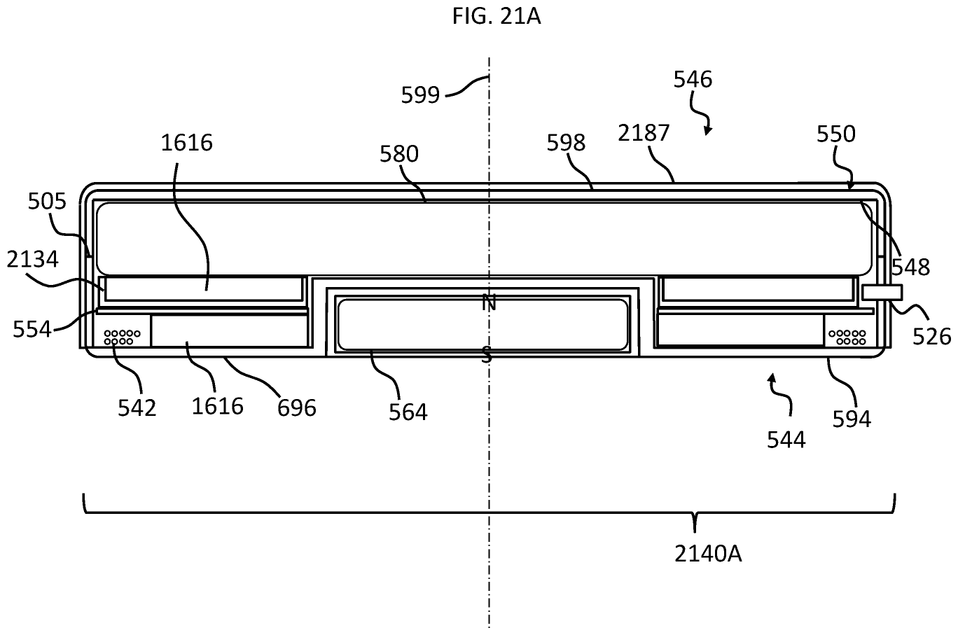

FIG. 20 provides an exemplary embodiment of a transcutaneous power transfer apparatus 2040 where the thermal mass 1919 is thermally insulated from the ambient environment by thermally insulative wall 2049 (and this embodiment shows battery 580 in direct contact with the thermal mass 1919). Here, the insulative wall 2049 limits heat transfer from the ambient environment into the thermal mass 1919 relative to that which would otherwise be the case. This results in an increase in the effectivity of the thermal mass 1919 relative to that which would exist if the wall 2049 was not present. (Some additional embodiments of this are described below.) FIG. 21 presents an exemplary embodiment of an external device 2140 where the internal thermal mass 1616 has thermal insulation 2134, which has a utility corresponding to the insulative wall 2049. It is noted that the wall that establishes the bottom skin interfacing surface 696 establishes a thermal insulation barrier for the bottoms of the bottom thermal masses 1616 in this embodiment. It is noted that in at least some exemplary embodiments, the housing can be thermally insulative housing (instead of a thermal mass).

FIG. 21A presents an exemplary external component 2140A that includes a thermal insulation barrier 2187 overlying the interior housing, where the interior housing is a thermal mass. Here, the thermal insulation barrier 2187 results in a thermally well-insulated external component. In an exemplary embodiment, closed-cell highly porous polymers can be utilized as the insulation barrier. This can be done for this embodiment and/or any of the other embodiments detailed above. As can be seen, the barrier 2187 extends around the sides and over the top of the external component, but not the bottom. In an exemplary embodiment, the bottom housing wall that is exposed to the ambient environment is not a thermal mass material, and thus there is no insulation over that component.

Thus, in view of the above, it can be seen that in some embodiments, the dedicated passive conduction thermal transfer apparatus is thermally insulated from an ambient environment (where “thermally insulated” means at least more than that which exists because of a general structure—a room in a house where the exterior wall(s) did not include insulation (e.g., the fluffy air trapping material placed between wall studs) would not be thermally insulated).

In an exemplary embodiment, the device is a headpiece for transcutaneous communication with an implantable hearing prosthesis inductance coil (or an implantable inductance coil of a vision prosthesis, or an implantable device that receives power via transcutaneous induction), and there are at least or equal to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 56, 57, 58, 59, 60, 61, 62, 63, 64, 65, 66, 67, 68, 69, 70, 71, 72, 73, 74, or 75 or more grams, or any value or range of values therebetween in 0.1 gram increments of dedicated thermal mass material in the device. In an exemplary embodiment, this is a metal. In an exemplary embodiment, these amounts do not include any portion of any magnet and/or any portion of any power storage device (e.g., such as the magnet), whether or not such may also be used as thermal mass in some embodiments (they are just not included in the totals). In an exemplary embodiment, the aforementioned mass values correspond to the mass of the structure that makes up the outer housing of the external component. In an exemplary embodiment, the aforementioned mass values correspond to the mass of the structure that makes up the portions of the housing of the external component that are located on the sides other than the skin interfacing side. In an exemplary embodiment, the aforementioned mass values correspond to the mass of the structure that can be seen from the outside of the external component. In an exemplary embodiment, the aforementioned mass values correspond to the mass of the structure that can be seen from the outside of the external component other than the portion on the skin interface side. In an exemplary embodiment, the aforementioned visible structures include the structure that is monolithic with the components that can be seen, as opposed to structure that is connected to those components on the inside, for example.

Thus, it can be seen that making a housing out of a metal or some other high thermal mass material can increase the mass of the housing relative to that which would otherwise be the case if the housing is made out of a polymer such as ABS.

In an exemplary embodiment, the dedicated passive conduction thermal transfer apparatus is configured to maintain the mean skin interface surface surface temperature below 25, 26, 27, 28, 29, 30, 31, 32, 33, 34, or 35 degrees C. for at least 5, 6, 7, 8, 9, 10, 11, 12, 13, 14, 15, 16, 17, 18, 19, 20, 21, 22, 23, 24, 25, 26, 27, 28, 29, or 30 minutes in a completely dark ambient air environment (the device is not exposed to thermal radiation beyond that associated with the ambient air temperature) of 35, 36, 37, 38, 39, or 40 degrees C. with the thermal mass being at 5, 6, 7, 8, 9, 10, 11, 12, 13, 14, or 15 degrees C. at the beginning of one or more of the aforementioned time periods, when the device is completely off and not being recharged.

In an exemplary embodiment, the device configured to maintain a mean and/or median and/or a total surface temperature of the thermal mass below 25, 26, 27, 28, 29, 30, 31, 32, 33, 34, or 35 degrees C. for at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 minutes in a completely dark ambient air environment of 35, 36, 37, 38, 39 or 40 degrees C. with the thermal mass being at 5, 6, 7, 8, 9, 10, 11, 12, 13, 14, or 15 degrees C. at the beginning of one or more of the aforementioned time periods, when the device is completely off and not being recharged.

It is noted that ambient air refers to the air around the device.

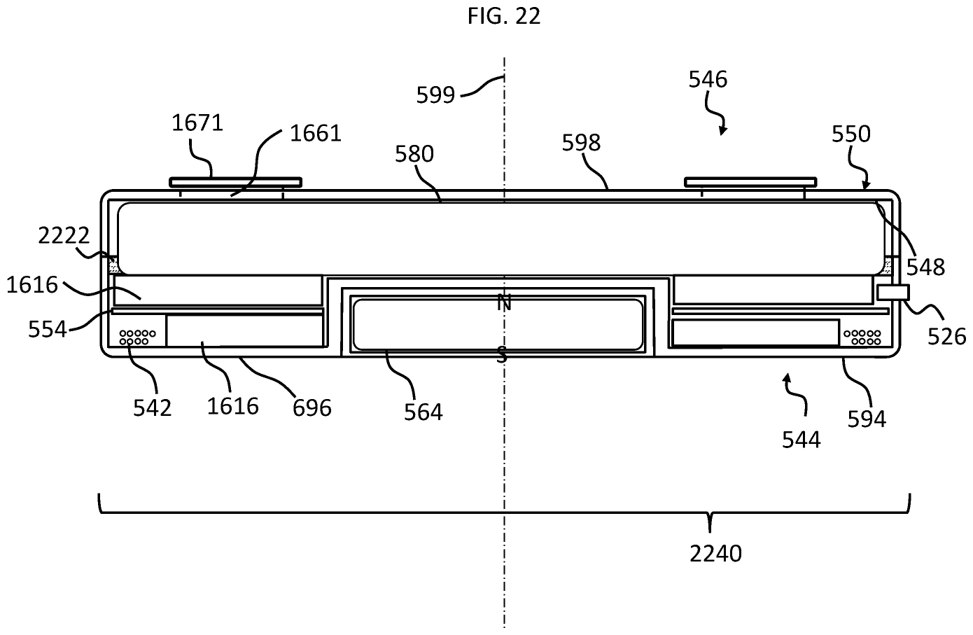

As noted above, in at least some exemplary embodiments, the external device is purposely designed so as to avoid or otherwise reduce heat transfer from the ambient environment to the thermal masses so as to keep the thermal masses at a lower temperature relative to that which would otherwise be the case, so as to absorb at least some of the heat from the power generating components. The corollary to this is that some embodiments may not otherwise include heat transfer devices used to transfer heat from inside the external component to the ambient environment because of the possibility that the reverse could happen. That said, in an alternative embodiment, insulation and heat transfer devices can be utilized in the context of an overall design that will permit heat transfer to the ambient environment while also shielding (or otherwise limiting the transfer to an acceptable overall amount—if the heat transfer to the thermal masses can be counterbalanced by the transfer to the ambient environment, were more utilitarianly, overshadowed, by such, such can be utilitarian) heat transfer from the ambient environment to the thermal masses. FIG. 22 presents an exemplary embodiment where thermal insulation 2222 is located around the periphery of battery 580 at the bottom of the battery 580, which insulation extends from the battery to the sidewalls of the housing as shown. As seen at the top, the transfer devices 1671 and 1661 permit conductive heat transfer to the services of component 1671, whereby convection, radiation and conductive heat transfer can take place to the ambient environment. Conversely, thermal masses 1616 located on the opposite side, and also the other side of the thermal insulation 2222. The idea here is that the battery itself and the installation 2222 will prevent or otherwise reduce the amount of the transfer from the ambient environment to the thermal masses. Thus, the thermal masses can be cooled or otherwise chilled and utilized in accordance with the teachings detail herein while also utilizing heat transfer techniques to the ambient environment.

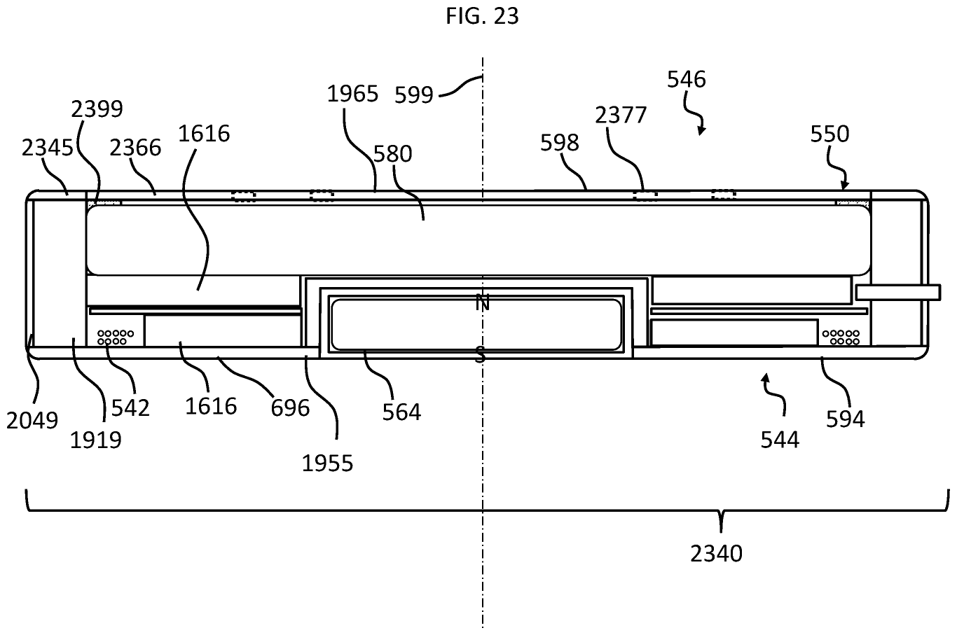

Referring back to FIG. 19, it is noted that instead of the housing being a thermal mass or otherwise made of metal or the like, a portion of the housing, such as housing wall 1919, can be made of a thermally insulated material. This cannot be utilitarian effect of insulating the thermal masses 1616. Indeed, again with reference to FIG. 20, the sidewalls of the housing adjacent the thermal mass 1919 can be insulated material. The thickness of the sidewalls can be adjusted as would be utilitarian. FIG. 23 depicts sidewalls 2049 that are thermally insulative, along with the bottom wall, where the top wall is designed to permit the transfer of heat from one side to the other, at least more than that which would be the case with respect to the sidewalls so as to encourage heat transfer from the battery 580 out the top surface. Here, the top wall is a composite wall where the portions above the thermal mass 1919, portions 2345, are thermally insulative, and the portion above the battery 580, portion 2366, is thermally conductive. That said, in an exemplary embodiment, the top of the housing can have holes 2377 or the like to permit airflow from inside the housing to outside the housing. In an exemplary embodiment, insulation portions 2399 can be utilized to establish a barrier between the thermal masses 1919 and the airspace above the battery 580 so that heat transfer will be limited from the thermal masses 1919 to that airspace relative to that which would otherwise be the case.

Still in some embodiments, in an exemplary embodiment, the entire housing can be thermally insulative.

It is noted that in some embodiments, the heat conductive properties of material utilized to prevent or limit heat transfer can be such that the material as resistivity to heat transfer that is 2, 2.5, 3, 3.5, 4, 4.5, 5, 5.5, 6, 6.5, 7, 7.5, 8, 9, 10, 11, 12, 13, 14, 15, 16, 17, 18, 19, 20, 25, 30, 35, 40, 45, 50, 55, 60, 70, 80, 90, or 100 times greater than material that is utilized to encourage heat transfer.

In an exemplary embodiment, the dedicated passive conduction thermal transfer apparatus includes a low temperature phase change material. By way of example, the material can be n-heneicosane. The material can be the material disclosed in U.S. Pat. No. 7,751,897. In an exemplary embodiment, the low temperature phase change material can be paraffin wax. In an exemplary embodiment, the material is a chemically inert, non-corrosive phase change material. The material can have a melting point of less than, greater than and/or equal to 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 51, 52, 53, 54, 55, 56, 57, 58, 59, 60, 61, 62, 63, 64, 65, 66, 67, 68, 69 or 70 degrees C. or any value or range of values therebetween in 0.1 degrees C. increments. The material can have a density of about 0.4, 0.5, 0.6, 0.7, 0.8, 0.9, 1.0, 1.1, 1.2, 1.3, 1.4, 1.5 grams per ml ready value or range of values therebetween 0.01 increments.

An exemplary embodiment, the phase changing material is utilized to fill void volume elements inside the external components, such as between gaps between individual parts and/or electronic components, etc.

In an exemplary embodiment, the material can be a material that absorbs heat or otherwise maintains a certain temperature while it is phase changing, in a manner analogous to boiling water. Thus, the dedicated passive conduction thermal transfer apparatus can be mass or otherwise a body or otherwise a vessel of this material. Indeed, in an exemplary embodiment, an elaborate piping system can be utilized that snakes or otherwise routes the material throughout various spaces within the external component. In this regard, in an exemplary embodiment, instead of and/or in addition to a discrete location where the material is located, material could be spread out throughout the interior of the external component, utilizing empty space that might otherwise be present. Of course, an exemplary embodiment, the aforementioned thermal mass can be arranged in a similar manner.

As seen above, an embodiment includes a dedicated passive conduction thermal transfer apparatus that is configured to absorb heat energy from the heat generating component(s) before that energy reaches the skin interface of the external component. Also, as can be seen, the apparatus absorbs heat from the heat generating component as opposed to absorbing heat of the skin interface. That is, in an exemplary embodiment, instead of cooling the skin interface surface, heat is prevented from reaching the skin interface surface.

In an exemplary embodiment, the active and/or passive cooling of the external component/charger as detailed herein and/or by cooled other means for that matter, can result in some instances where the temperature goes below the prevailing local dewpoint at the location of the charger/external component. This can lead to condensation resulting from the ambient humidity, such as may be the case in a non-controlled RH environment. By way of example only and not by way of limitation, in an exemplary embodiment, cooling the external components to a temperature of, for example, 10° C., where the local dewpoint is 20° C., can result in the condensation just detail. In this regard, in an exemplary embodiment, the charger/external component can act as a nucleation point for condensing the ambient moisture.

In at least some exemplary embodiments, there can be utilitarian value with respect to wiping down the charger/drying the charger, prior to using the external component to recharge the implanted component and/or during the action of recharging the implanted component. That said, in some other embodiments, there can be utilitarian value with respect to utilizing this moisture in an evaporative cooling regime. By way of example only and not by way of limitation, in an exemplary embodiment, the housing and/or otherwise the outer surfaces of the external component/charger, can be three-dimensionally structured in such a way that the surface of the charger/external component collects or otherwise pools the moisture, and in some embodiments, contains the moisture at a location where there can be utilitarian value with respect to utilizing that moisture for evaporative cooling. (Note that this is distinguished from the phase changing material detailed above—with respect to the phase changing, the material is repeatedly reused/trapped within or otherwise as a part of the external component. This is contrasted to the utilization of moisture from the ambient environment or from an external source, which will be a wasting asset with respect to the point in time where the moisture is completely evaporated.)

For example, in an exemplary embodiment, there can be channels on the surface of the external component, such as those that can be seen from the outside when the external component is attached to the head of a recipient, which channels utilize gravity when the device is being utilized to charge the implanted component to channel the moisture into collection volumes, which can be miniature tanks or can be open reservoirs or like components on the outside of the external component (or can be located inside the external component, in fluid communication with the outside environment via a channel that extends to the housing, by way of example only and not by way limitation). That said, in an alternate embodiment, hydrophilic surfaces can be used on the external housing (and/or on the inside, for that matter). Conversely, there can be utilitarian value with respect to utilizing a hydrophobic surface that corresponds to the skin interface surface. This latter feature can be used in any embodiment irrespective of whether or not evaporative cooling is going to be utilized.

In any event, the moisture is collected or otherwise stabilized (utilizing a hydrophilic surface may not necessarily collect the moisture/corral the moisture—instead, the moisture is simply retained or otherwise impeded from moving in a significant manner via the hydrophilic surface), the evaporative cooling that takes place as the moisture evaporates can be utilized to limit an increase of the temperature of the charging device relative to that which would otherwise be the case in the absence of the evaporative cooling.

In some embodiments, the evaporative cooling occurs without any additional heat transfer infrastructure. For example, the scenario where the outer surface of the housing of the external component is coated in moisture or otherwise has an increased level of moisture thereon relative to that which would otherwise be the case is utilized just as it is. Conversely, heat pipes or a dedicated heat transfer arrangement can be utilized to enhance the heat transfer from the heat generating components to the location where evaporative cooling is taking place. Indeed, in an exemplary embodiment, referring to FIG. 17, the cap 1777 can be placed over the outer housing, thus trapping the moisture that has accumulated on the housing at the surface opposite the skin interface surface, and the heat transfer components 1671 can be utilized to transfer the heat to the moisture. This can have the dual utilitarian value of insulating the external component so that the rate of increase in temperature thereof is lower than that which would otherwise be the case owing to the cap, while also taking advantage of the evaporative cooling that occurs. Steam vents or the like can be placed in the cap.

In any event, in at least some exemplary embodiments, there are components that channel heat and otherwise conduct heat generated during the recharging process to the evaporatively cooled surfaces and/or bodies, etc. These components are specifically designed and otherwise provided in the external component for such purpose.

In another exemplary embodiment, a reservoir or a tank can be located in the interior of the housing, which tank collects moisture, and where during evaporation, evaporative cooling takes place.

In an exemplary embodiment, the reservoir or tanks can be located within a housing wall—that is, the housing can be hollow in some locations. This can have further utilitarian value with respect to insulation—where once the moisture has evaporated, an air pocket will remain within the housing wall, which further insulates the external component. In fact, in some embodiments, a check valve or the like can be utilized so that after the majority and/or the vast majority and/or all of the moisture has evaporated, and exited out the check valve, the check valve will then secure the air within the hollow portion, further enhancing the insulative features.

Indeed, the air pocket concept can be used in any embodiment, irrespective of whether evaporative cooling is used. FIG. 21 shows an example of an air pocket 2171 in the housing wall that is opposite the skin interface surface. It is noted that in at least some exemplary embodiments, the air pockets can be located at other locations in an alternative format and/or in addition to what is shown in FIG. 21 and/or the sizes of the air pockets can be varied. In an exemplary embodiment, the air pockets can be located on the sidewalls. Any arrangement that can have utilitarian value of the respect to enhancing the insulative features of the housing can utilize at least some exemplary embodiments if such as utility. Embodiment, the air pockets are hermetically sealed after they are established. In other exemplary embodiments, the air pockets can be controllably opened and or sealed depending on the insulative effect desired. In this regard, in an exemplary embodiment, the opening/sealing can be executed manually and/or can be controlled utilizing onboard circuitry, depending on the utilitarian value with respect to the insulative properties associated with the air pocket. Again, a valve can be utilized to control the opening/sealing.

Returning back to the evaporative cooling embodiments, in some exemplary embodiments, hydrophobic surfaces can be utilized on the channels so that the moisture will flow more easily or otherwise more readily to the locations where it is utilitarian to collect the moisture, such as in the aforementioned tanks or reservoirs. That said, as noted above, extensive use of hydrophilic surfaces can utilize simply to increase the amount of moisture located on the external portions of the housing where it is utilitarian to have that moisture relative to that which would otherwise be the case in the absence of the hydrophilic surface utilization.

In at least some exemplary embodiments, there are external components that have a surface that is treated or otherwise made out of a hydrophilic substances. Embodiments include the utilization of materials and/or coatings that results in moisture retention beyond that which is the case with respect to the utilization of the polymers detailed herein and/or the metals detailed herein.

An exemplary embodiment, moisture retention on a per unit basis is at least and/or equal to 30, 50, 70, 90, 125, 150, 175, 200, 250, 300, 350, 400, 500, 600, 700, 800, 900, 1000 percent on a per unit basis or any value or range of values therebetween in 1% increments greater than that which results from the utilization of polymers and/or the metals detailed herein for the surface, all other things being equal, and in some embodiments, this is achieved at any one or more of the aforementioned cooling temperatures associated with the external component in any one or more of the ambient environment regimes detailed herein, where the relative humidity is at least and/or equal to and/or no more than 50, 55, 60, 65, 70, 75, 80, 85, 90, 95 or 100% or any value or range of values therebetween in 1% increments.

In an exemplary embodiment, for any one or more of the actions detailed herein, evaporative cooling constitutes at least and/or equal to 5, 10, 15, 20, 25, 30, 35 or 40% or more or any value or range of values therebetween in 1% increments of the cooling/temperature increase limiting regimes in at least some embodiments.

In another exemplary embodiment, a moisture generating source can be provided with the dedicated chargers. In this regard, and an exemplary embodiment, a moisture rich environment can be provided in the receptacle of the chargers. In an exemplary embodiment, this arrangement can be controlled utilizing the circuitry and/or processors of the dedicated charger in order to control the magnitude of the evaporative cooling effect that will be implemented with a precooled charger. In an exemplary embodiment, the dedicated recharger can be configured to apply moisture to certain surfaces of the external component. In another exemplary embodiment, the reservoirs/tanks can be purposely charged with water. In this regard, in an exemplary embodiment, the dedicated charger could have a hose or other device that provides water to the external component.

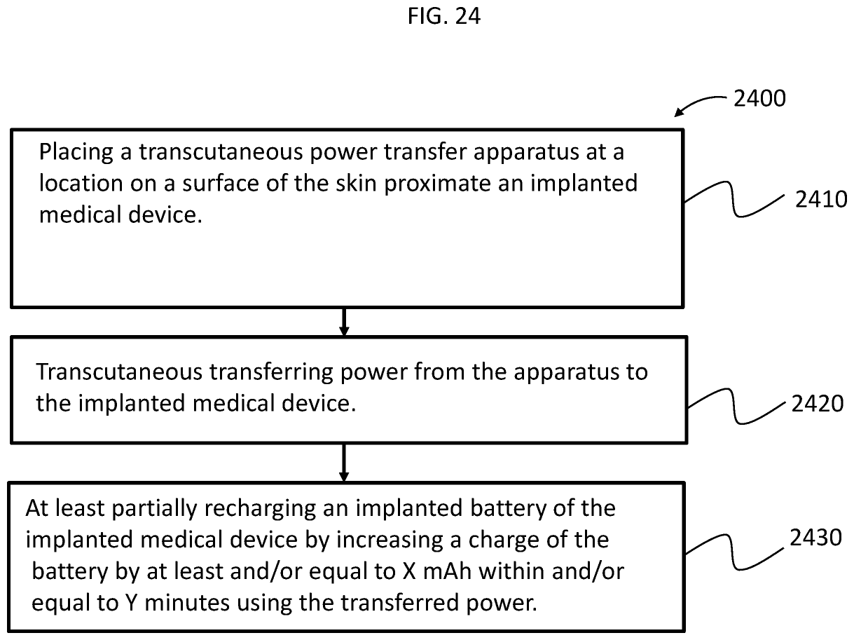

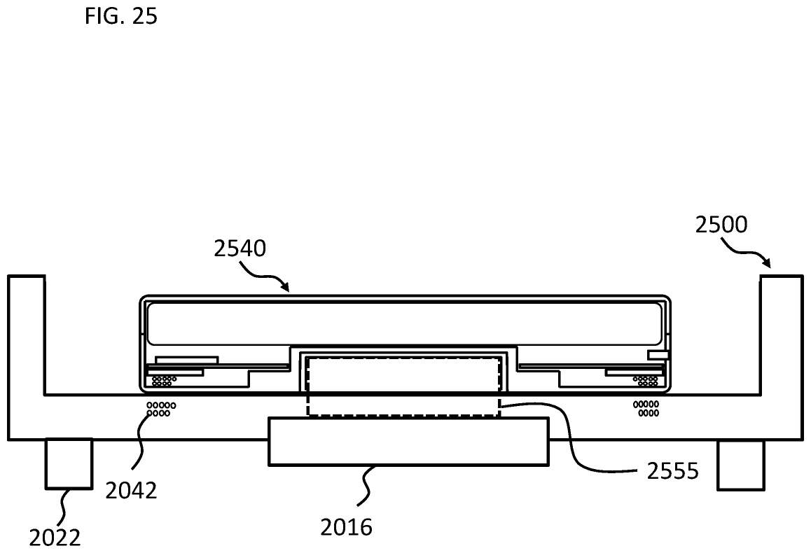

FIG. 24 presents an exemplary flowchart for an exemplary method, method 2400, which includes method action 2410, which includes placing a transcutaneous power transfer apparatus at a location on a surface of the skin proximate an implanted medical device. Method 2400 also includes method 2420, which includes transcutaneously transferring power from the apparatus to the implanted medical device. Method 2400 also includes method action 2430, which includes at least partially recharging an implanted battery of the implanted medical device by increasing a charge of the battery by at least and/or equal to X mAh within and/or equal to Y minutes using the transferred power, wherein X is 5, 6, 7, 8, 9, 10, 11, 12, 13, 14, 15, 16, 17, 18, 19, 20, 21, 22, 23, 24, 25, 26, 27, 28, 29, 30, 35, 40 or more, or any value or range of values therebetween in 0.1 increments, and Y is 5, 6, 7, 8, 9, 10, 11, 12, 13, 14, 15, 20, 25, 30, 35, 40, 45, 50, 55, 60, 70, 80, 90, 100, 110, 120, 130, 140, 150, 160, 170, or 180 in 0.1 increments.

Consistent with the teachings detailed above, in some exemplary embodiments method 2400 can be executed utilizing a transcutaneous power transfer apparatus having a transmitting inductance coil, where the implanted medical device includes an inductance coil (receiving coil), although it is noted that in some embodiments, the coils can also communicate in the opposite direction, or more accurately, the components can have receivers and transceivers and transmitters that can permit two-way communication. In an exemplary embodiment, coils have a maximum outer diameter no more than 40, 35, 30, 25, or 20 mm, or any value or range of values therebetween in 1 mm increments (and the coils need not have the same outer diameters, but can have such). In an exemplary embodiment, consistent with the teachings above, the implanted coil is located completely above the mastoid bone of a human, beneath the skin, while in some embodiments, the coil can be located in an excavation within the mastoid bone.

In an exemplary embodiment, the transmitting inductance coil of the apparatus is no closer than 20, 19, 18, 17, 15, 15, 14, 13, 12, 11, 10, 9, 8, 7, 6 or 5 mm from the implanted receiver inductance coil.

In an exemplary embodiment, the battery of the implant is a lithium-ion battery, which battery has a 10, 15, 20, 21, 22, 23, 24, 25, 26, 27, 28, 29, 30, 31, 32, 33, 34, 35, 40, 45 or 50 or more mAh power rating or any value or range of values therebetween in 0.1 imAh increments. In an exemplary embodiment, no more than or equal to 70, 75, 80, 85 or 90% or any value or range of values therebetween in 1% increments of the nominal capacity (the just detailed numbers) of the battery is used. For example, with a nominal 25 mAh battery, at 80% usage, the charging will be 20 mAh. In an exemplary embodiment, the voltage associated with the charging is 1, 2, 3, 4, 5, 6, 7, or 8 volts or any value or range of values therebetween in 0.1 V increments.

In an exemplary embodiment, the efficiency of the power link is less than, equal to or greater than 20, 21, 22, 23, 24, 25, 26, 27, 28, 29, 30, 31, 32, 33, 34, 35, 36, 37, 38, 39, 40, 45 or 50%, or any value or range of values therebetween in 1% increments. In an exemplary embodiment, the charge current at the battery terminals is the nominal capacity used divided by the length of time charging. So, for example, if the nominal capacity is 20 mAh, and charging takes place for 5 minutes, the charging current would be thus 240 mA (which is about 1 Watt at 4 Volts). The charge current at the transmission coil terminal with an efficiency of 30% would be, for example, 1 Watt/30%=800 mA (which is about 3.2 Watts). This results in losses of 2.2 Watts plus the additional loss inside the charger itself.

In an exemplary embodiment, there is an action of at least partially recharging an implanted battery of the implanted medical device by increasing a charge of the battery by at least 10 mAh within 10 minutes using the transferred power. This can correspond to at least partially recharging the implanted battery of the implanted medical device by increasing a charge of the battery by at least 10 mAh within 5 minutes using the transferred power. This can also be part of a method that includes at least partially recharging the implanted battery of the implanted medical device by increasing a charge of the battery by at least 20 mAh within 7 minutes using the transferred power, or at least partially recharging the implanted battery of the implanted medical device by increasing a charge of the battery by at least 20 mAh within 5 minutes using the transferred power.

Thus, as seen above, in exemplary embodiments, scenarios exist where an implanted lithium ion battery can be fully charged within five minutes, where the charger and the transmission coil heating due to the losses are managed according to the embodiments detailed herein by way of example.

And by way of management, in an exemplary embodiment, the mean, median, and/or mode and/or maximum surface temperature at any location on the skin interface surface during the time of charging is not the above one or more of the various temperatures detailed herein, such as, for example, as will be detailed now.

The teachings detailed herein can enable the utilization of charging techniques and/or power techniques in relatively high ambient environment temperatures, such as for example, during a heat wave in the southeast or southwest United States, where a recipient of the prosthesis is outside, and at least outside for an extended period of time. In an exemplary embodiment, the methods and devices and systems are used in an ambient environment in the shade or in sunlight after a recipient and/or the external device has been in that environment for at least 1, 1.5, 2, 2.5 or 3 hours prior to commencement of power transfer, where the ambient environment temperature is above 35, 36, 37, 38, 39, 40, 41, 42, 43, 44, or 45 degrees Celsius at the time that power transfer is commenced and has been such for at least 0.5, 1, 1.5, 2, 2.5, 3, 3.5, 4, 4.5, 5, 5.5, 6, 7, or 8 hours, prior thereto.

In this regard, a temperature ambient the location at the skin of recharging is above any of the aforementioned temperatures for at least 0.5 1, 1.5, 2, 2.5, 3, 3.5, 4, 4.5, 5, 5.5, 6, 7, or 8 hours, before commencement of the action of charging and the temperature at the location is maintained at below 41 degrees Celsius (mean, median and/or mode and/or at any given location) during the entire time of transferring power from the apparatus to the medical device, which time corresponds to the time to get a given of a percentage of a nominal capacity using the above noted parameters, and within the above noted time periods.

Some exemplary embodiments include executing one or more of the implant charging actions detailed herein while maintaining a skin temperature at the location of power transfer below 43, 42, 41, 40, 39, 39, 37, 36 or 35 degrees Celsius for the entire time that charging is executed. In an exemplary embodiment, any one or more of the method actions detailed herein begins with a skin temperature, prior to contact of the external device to the skin of the recipient, that is less than greater than or equal to 29, 30, 31, 32, 33, 34, 35, 36, 37 or 38 degrees Celsius or any value or range values therebetween in 0.1° increments. Starting from this skin temperature, the method actions detailed herein can be executed such that the implant is recharged while maintaining the skin temperature at the aforementioned temperatures at the beginning of this paragraph.

It is noted that the above noted temperature for the skin can also be for the skin interface surface of the external device, and can be mean, median and/or mode and/or for any part of the surface facing the skin. Disclosure of one corresponds to a disclosure of the other, and vice versa.

In an exemplary embodiment, the teachings detailed herein can be utilized to increase a rate of charge of charging of an implanted component/implanted battery using the external component. In this regard, in an exemplary embodiment, utilizing a device without the cooling/chilling actions detailed herein might result in a temperature of skin of the recipient and/or the skin interface surface reaching 39, 40, 41, 42, or 43 degrees Celsius or more during the charging operation. These temperatures can be dangerous and/or otherwise uncomfortable. The recipient of the prosthesis that is being charged might be inclined to stop the charging process because the heating of the skin is uncomfortable. That said, in some embodiments, the device may automatically shut off or otherwise stop charging or otherwise reduce the rate of charging because the device senses that the skin temperature is being raised to a unacceptable and/or undesirable level (either by a direct skin temperature sensor that is part of the external component were utilizing a latent variable to detect such, such as that which may be the case utilizing a sensor that detects the temperature of a portion of the external component and extrapolates or otherwise deduces an estimated temperature of the skin). Accordingly, in at least some exemplary embodiments, by utilizing the cooling techniques detailed herein, that skin temperature and/or the skin interface surface will not reach these unacceptable or otherwise uncomfortable temperatures, and thus the external component can be used for a longer period of time to charge the implant (while maintaining the charging rate that existed, say, for at least, 50, 55, 60, 65, 70, 75, 80, 85, 90, 95, or 100% of the previous charging time during the charging period—as opposed to reducing the rate to reduce the temperature), thus enabling the implant to be charged “more” and/or more quickly than that which would otherwise be the case, all other things being equal.

Indeed, in an exemplary embodiment, the actions of charging the implant are executed such that during a time of recharging, a rate of charging does not deviate more than 2, 3, 4, 5, 6, 7, 8, 9, 10, 15, 20, 25, or 30% from an average rate of charge of a charging process (mean, median and/or mode), excluding ramp up or ramp down periods that are utilized for battery life preservation, etc., for more than 5, 10, 15, 20, 25, or 30% of the total time that the external device is against the skin of the recipient. Accordingly, in an exemplary embodiment, there are methods of charging where the transmitted power is not purposely powered down or otherwise limited for temperature reasons (it might be limited for other reasons).

In an exemplary embodiment, the methods detailed herein can be executed 10, 15, 20, 25, 30, 35, 40, 50, 60, 70, 80, 90, 100, 125, or 150 times or more with the same device(s) while meeting the parameters detailed herein.

As noted above, there can be utilitarian value with respect to cooling the headpiece at least of the external component that is utilized to charge the implanted component relative to that which would otherwise be the case at the commencement of charging of the implanted device. Here, in this embodiment, the external component would start off at a lower temperature relative to that which would otherwise be the case owing to ambient conditions, and thus the heat generated as a result of the recharging of the external component would be “taken up” by the fact that the external component starts off colder than that which would otherwise be the case.

As promised above, we now return to the device 2000 of FIG. 14. Device 2000 is a device comprising a battery charging apparatus (e.g., a system having an inductance communication device in communication with a transformer and other circuitry that can be powered by a battery or by alternating current from a household power outlet), and a cooling device, which can be a refrigeration system or a convection device (e.g., the fan 2002), all by way of example. The device is a dedicated prosthesis component charging device configured to recharge a power storage portion of the prosthesis component before and/or after cooling (including chilling) an assembly of which the power storage portion is apart using the cooling device. With respect to the embodiment under explanation, the device is a dedicated hearing prosthesis component charging device.

In an exemplary embodiment, the device is configured to recharge a power storage portion of the prosthesis component. The power storage portion can be battery cells that are configured to be recharged. In an exemplary embodiment, the prosthesis component is a battery, such as battery 252 of the BTE device 1040 of FIG. 4. In an exemplary embodiment, the prosthesis component is an external component of a hearing prosthesis in its entirety (e.g., component 640 as seen in FIG. 14), and in another exemplary embodiment, it is the prosthesis charging device (in its entirety), such as the device used to recharge a totally implantable hearing prosthesis, such as for example the variation of the embodiment of FIG. 6 (where there is no sound capture element 526 and no sound processor—the device is purely a device configured to recharge the implanted portion) or FIG. 11 (by way of example only). The device is configured to cool an assembly of which the power storage portion is apart (e.g., the assembly of battery 252, the assembly of the off the ear charger, etc.).

Some embodiments of the charging device are configured to charge one or more of the aforementioned external components and/or batteries detailed herein in accordance with one or more of the various recharging regimes detailed herein. The charging device 2000 can be configured to plug into a standard alternating current outlet so as to obtain power for the operation of the charging device. The charging device 2000 is configured with a lid 2020 that enables the inside of the charging apparatus to be isolated from the ambient environment. This can have utilitarian value with respect to this particular embodiment in which the charging device 2000 is unique in that the charging device also enables cooling of the external component 640 during charging and/or while the external component is located therein. In this regard, the exemplary charging device 2000 seen in FIG. 24 includes three thermoelectric coolers 2016. As seen, two of these have heatsinks 2061 that lead to radiator devices 2071 so as to transfer heat from inside the enclosure to outside the enclosure, thus cooling the external component 640. The thermoelectric cooler 2016 at the bottom is seen extending all the way through the enclosure wall and does not have a heatsink per se. As seen, support pedestals 2022 are located on the bottom of the charging device 2000 so that air can flow under the bottom of the thermoelectric cooler 2016. To be clear, in the embodiment shown in FIG. 20, the “cool side” of the thermoelectric coolers are located facing the external component 640, and the “hot side” of the thermoelectric coolers are located facing the outside of the enclosure, away from the external component. This enables heat to be transferred from the external component or otherwise from the inside of the enclosure to the outside of the enclosure, thus effectively refrigerating the inside of the charging device.

Also as seen, the charging device of FIG. 20 includes a fan 2002 which can be utilized to transfer heat from the external component 640. In an exemplary embodiment, the charging apparatus can be configured with inlet and outlet ports so that airflow through the enclosure can be enhanced or otherwise enabled. That said, in an alternate embodiment, such as where the enclosure is in a semi-sealed configuration (akin to how a refrigerator enclosure is sealed), the fan 2002 can be utilized to move air within the enclosure so that there is airflow across the “cold sides” of the thermoelectric cooling devices.

It is noted that while the charging device of FIG. 14 relies on thermoelectric cooling and/or convection heat transfer, in an alternative embodiment, a refrigeration system utilizing compressed and expanded gas (the Carrier refrigeration cycle) can be utilized instead or in addition to the embodiments seen in FIG. 14. Note also that in some embodiments, a more technologically simple arrangement might be utilized external component. In an exemplary embodiment, pre-cooled substances, such as an ice pack, can be placed in the enclosure to cool the enclosure and otherwise extract heat from the external component 640. In an exemplary embodiment, this icepack can be a preformed component (e.g., it is not a bag of ice, but instead a plastic container containing a substance that is easily cooled in a repeatable manner) that can be put in a freezer or otherwise maintained in a freezer and then taken out and utilized when recharging is to be implemented. In an exemplary embodiment, the icepack can be placed on top of the lid, and the lid can have a preformed structure that can receive the icepack, and air vents through the lid can suck air from outside the enclosure into the enclosure, which air will be cooled while the air passes over/around the icepack, thus drawling cold air into the enclosure, and thus cooling the external component 640 during charging.

Thus, in an exemplary embodiment, there is a method including one or more of the actions herein that also include the additional action of obtaining access to a charging apparatus configured to interface with the device to be recharged (the external component) and recharge the power storage component, wherein the action of recharging the power storage component is executed using the charging apparatus that is configured to affirmatively cool the device during the charging and/or before the charging, and the charging apparatus is used to affirmatively cool the device. Here, the charging apparatus to which access is obtained can be the apparatus of FIG. 20. Thus, in some embodiments, the charging apparatus to which access is obtained includes a container. In this regard, the methods can further include the method actions of placing the device to be recharged into the container so that the device is completely enclosed in the container, and lowering the temperature of the device while the device is in the container.

In some embodiments, the interior of the container is cooled to a temperature of or below 33, 32, 31, 30, 29, 28, 28, 27, 26, 25, 24, 23, 22, 21, 20, 19, 18, 17, 16, 15, 14, 13, 12, 11, 10, 9, 8, 7, 6, 5, 4, 3, 2, 1, −1, −2, −3, −4, −5, −6, −7, −8, −9, −10, −11, −12, −13, −14 or −15 degrees Celsius or lower, and such is the case for at least 2, 3, 4, 5, 6, 7, 8, 9, 10, 11, 12, 13, 14, 15, 20, 25, 30, 35, 40, 45, 50, 60, 70, 80, 90, 100, 110, 120, 130, 140, 150, 160, 170, or 180 minutes or longer, all while the device being charged is in the container being charged. Note that the method claims can include using a device, such as a refrigerator and/or freezer, to achieve these temperature ranges.

In essence, device 2000 can be considered a cooling box that can contain the entire charger (the transcutaneous power transfer device), and has a power supply to provide power to the charger, and cooling functions, is insulated to maintain a low temperature (e.g., 10° C.) inside the receptacle when it is closed, and can have a Peltier element (or other cooling mechanism) and optionally also a fan in order to provide cooling when the lid is closed, and a recharge interface to recharge the battery inside the charger.

The recharge interface can be a lead that connects to the charger, or it can be a wireless charging coil situated inside the cooling box so it matches the wireless charging coil associated with recharging the battery inside the charger. Alternatively, the battery inside the charger may be replaced with a fully charged one. The charger is a specific example of a highly integrated charger that contains a battery, electronics that allow to recharge the battery inside the charger or a mechanical facility (e.g. movable and/or removable lid) that allows replacing the battery inside the charger, electronics and a transmit coil that allow wirelessly recharging a target device with a matching receiver coil, and an alignment facility (e.g., adjustable magnet) that permits charger and target device coils to be aligned.

FIG. 21 presents another exemplary embodiment of a charger 2500, for an external component, here, external component 2540. In this embodiment, charger 2500 includes a heatsink 2555 that extends from the thermoelectric cooler 2016 as shown. The heatsink 2555 is sized and dimensioned to fit into the external component 2540 as shown. In this regard, the external component includes a coupling to removably attached to the external component to a separate heat transfer device. This coupling interfaces with the heatsink 2555. He can be transferred from the external component 2540, including from inside the external component, directly to the heatsink 2555. This can enhance heat transfer during recharging. This heat transfer can be executed during recharging. In an exemplary embodiment, the magnet can be removed from the external component 2540 to provide access to the coupling.

It is noted that the external components without the heat transfer systems/cooling systems/chilling systems/the dedicated passive conduction thermal transfer apparatuses do not include part of the specification, and should be considered prior art. Thus, embodiments include means for inductance power transfer communication, which include for example an inductance coil established by heat pipes, and because the traditional/prior art inductance power transfer communications do not form part of the innovative features, but are modified with innovative features herein/are used with the innovative features, means for inductance power transfer communication does not include these prior art devices. Such is also the case with respect to heat transfer deices/apparatuses/systems—the mere fact that any device may transfer heat does not corresponds to a heat transfer device, etc.

In an exemplary embodiment, the devices that are dedicated prosthesis charging device are configured such that when they are inductively coupled to a prosthesis charging component for inductive charging of that component, the devices can fully recharge the component from at least a 50, 55, 60, 65, 70, 75, 80, 85, 90, 95 or 100% depleted battery state in a time period that is at least 20, 25, 30, 35, 40, 45, 50, 55, 60, 65 or 70% or any value or range of values therebetween in 1% increments shorter than that which would otherwise be the case in the absence of one or more of the teachings herein, all other things being equal, such that the skin interface component is not above 37, 38, 39, 40, 41, 42, or 43 degrees Celsius at the end of the time period when an ambient temperature of the device is at least 35, 36, 37, 38, 39, 40, 41 or 42 degrees Celsius or any value or range of values therebetween in at least 0.1° C. increments in a shaded still air condition.

In an exemplary embodiment, the charging devices include circuitry, such as microprocessors, configured to implement fast charging versus standard charging (they are configured to implement both—the devices are configured to utilize the circuitry so as to implement such charging regimes).

It is noted that any method detailed herein also corresponds to a disclosure of a device and/or system configured to execute one or more or all of the method actions associated with the device and/or system as detailed herein. In an exemplary embodiment, this device and/or system is configured to execute one or more or all of the method actions in an automated fashion. That said, in an alternate embodiment, the device and/or system is configured to execute one or more or all of the method actions after being prompted by a human being. It is further noted that any disclosure of a device and/or system detailed herein corresponds to a method of making and/or using that device and/or system, including a method of using that device according to the functionality detailed herein.

It is further noted that any disclosure of a device and/or system detailed herein also corresponds to a disclosure of otherwise providing that device and/or system.

It is also noted that any disclosure herein of any process of manufacturing and/or providing a device corresponds to a device and/or system that results therefrom. It is also noted that any disclosure herein of any device and/or system corresponds to a disclosure of a method of producing or otherwise providing or otherwise making such.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. A method, comprising:
placing a transcutaneous power transfer apparatus at a location on a surface of the skin proximate an implanted medical device;
transferring power from the apparatus to the implanted medical device; and
actively cooling the transcutaneous power transfer apparatus below the ambient temperature prior to and/or after commencing transfer of power from the apparatus to the implanted medical device.

2. The method of claim 1, wherein:
the apparatus includes a dedicated heat transfer arrangement configured to transfer away from the apparatus heat so as to cool the apparatus, and the dedicated heat transfer arrangement is used to actively cool the apparatus in the cooling action.

3. The method of claim 1, wherein:
the action of cooling the transcutaneous power transfer apparatus below the ambient temperature is executed prior to commencing transferring power from the apparatus to the implanted medical device.

4. The method of claim 1, wherein:
the action of cooling the transcutaneous power transfer apparatus below the ambient temperature is executed after commencing transferring power from the apparatus to the implanted medical device.

5. (canceled)

6. The method of claim 1, wherein:
the action of transferring power is executed as part of a fast charge of the implant, wherein heat generated by the apparatus as a result of the fast charge is absorbed by a heat absorbing arrangement of the apparatus, thereby preventing a skin interface surface of the apparatus from exceeding a temperature beyond that which would otherwise be the case, which temperature would be at least uncomfortable for the recipient.

7. The method of claim 1, wherein:
the action of cooling is not executed during the action of transferring power, wherein the action of transferring power raises a state of charge of a power storage apparatus of the implant by at least 50% or to at least 50%.

8-18. (canceled)

19. A device, comprising:
an inductive power transmission system configured to transfer power to an implanted medical device;
a skin interface surface; and
a dedicated passive conduction thermal transfer apparatus configured for temperature management of the device during the transfer of power.

20. The device of claim 19, wherein:
the dedicated passive conduction thermal transfer apparatus is a dedicated thermal mass configured for thermal mass cooling of the device.

21. The device of claim 19, wherein:
the dedicated passive conduction thermal transfer apparatus is thermally insulated from an ambient environment.

22. The device of claim 20, wherein:
the device is a headpiece for transcutaneous communication with an implantable hearing prosthesis inductance coil; and
there are at least 15 grams of dedicated thermal mass material.

23. The device of claim 20, wherein:
the device is a headpiece for transcutaneous communication with an implantable hearing prosthesis inductance coil; and
there are at least 30 grams of dedicated thermal mass material.

24. The device of claim 19, wherein:
the dedicated passive conduction thermal transfer apparatus includes a low temperature phase change material.

25. The device of claim 20, wherein:
the dedicated passive conduction thermal transfer apparatus is configured to maintain, when the device is completely off and not being recharged, the mean skin interface surface surface temperature below 30 degrees C. for at least 15 minutes in a completely dark ambient air environment of 40 degrees C. with the thermal mass being at 5 degrees C. at the beginning of the 15 minutes.

26-33. (canceled)

34. A device, comprising:
a battery charging apparatus; and
a cooling device, wherein
the device is a dedicated prosthesis component charging device configured to recharge a power storage portion of the prosthesis component before and/or after cooling an assembly of which the power storage portion is apart using the cooling device.

35. The device of claim 34, wherein:
the device is a dedicated hearing prosthesis component charging device.

36. The device of claim 34, wherein:
the device includes a means for cooling before and/or after cooling an assembly.

37. The device of claim 34, wherein:
the device is a fast charger for an implant charger for a totally implantable sensory prosthesis.

38. The device of claim 34, wherein:
the device includes a compartment sized and dimensioned to receive the prosthesis component; and
the device is configured to reduce an air temperature within the compartment by at least 5 degrees Celsius relative to an ambient air temperature of air in which the device is located.

39. The device of claim 34, wherein:
the prosthesis component is inductively coupled to the device; and
the device is configured to fully recharge the prosthesis component from at least a 90% depleted battery state in a time period that is at least 30% shorter than that which would otherwise be the case in the absence of the availability of the functionality of the cooling device, all other things being equal, such that the skin interface component is not above 41 degrees Celsius at the end of the time period when an ambient air temperature of the device is at least 35 degrees Celsius in a shaded still air condition.

40. The device of claim 34, wherein:
the device is configured to expose the prosthesis component to humidity different than that which would otherwise be the case vis-à-vis an ambient environment.

41-43. (canceled)
